SEQUENCE LISTING (28 sequences)

SEQ ID NO: 1 - protein, 74 aa, Homo sapiens
MLGLVLALLSSSSAEEYVGLSANQCAVPAKDRVDCGYPHVTPKECNNRGCCFDSRIPGVPWCFKPLTRKTECTF

SEQ ID NO: 2 - protein, 73 aa, Homo sapiens
MLGLVLALLSSSSAEEYVGLSANQCAVPAKDRVDCGYPHVTPKECNNRGCCFDSRIPGVPWCFKPLQEAECTF

SEQ ID NO: 3 - protein, 80 aa, Homo sapiens
MAARALCMLGLVLALLSSSSAEEYVGLSARGCAVPAKDRVDCGYPHVTPKECNNRGCCFDSRIPGVPWCFKPLQEAECTF

SEQ ID NO: 4 - protein, 130 aa, Homo sapiens
MQERTGAATARRESLPQANNPEQLCKQRCINEASWTMKRVLSCVPEPTVVMAARALCMLGLVLALLSSSSAEEYVGLSANQCAVPAKDRVDCGYPHVTPKECNNRGCCFDSRIPGVPWCFKPLQEAECTF

SEQ ID NO: 5 - DNA, 398 bp, Homo sapiens
gatgctggggctggtcctggccttgctgtcctccagctctgctgaggagtacgtgggcctgtctgcaaaccagtgtgccgtgccggccaaggacagggtggactgcggctacccccatgtcacccccaaggagtgcaacaaccggggctgctgctttgactccaggatccctggagtgccttggtgtttcaagcccctgactaggaagacagaatgcaccttctgaggcacctccagctgcccctgggatgcaggctgagcacccttgcccggctgtgattgctgccaggcactgttcatctcagtttttctgtccctttgctcccggcaagctttctgctgaaagttcatatctggagcctgatgtcttaacgaataaaggtcccatgctccacccg

SEQ ID NO: 6 - DNA, 685 bp, Homo sapiens
gccaaaacagtgggggctgaactgacctctcccctttgggagagaaaaactgtctgggagcttgacaaaggcatgcaggagagaacaggagcagccacagccaggagggagagccttccccaagcaaacaatccagagcagctgtgcaaacaacggtgcataaatgaggcctcctggaccatgaagcgagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctgaaaaaaaaaaaaaaaaaa

SEQ ID NO: 7 - DNA, 491 bp, Homo sapiens
ggagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccaccctaaaaaaaaaaaaaaaaaaaaaaaaaaaaaa

SEQ ID NO: 8 - DNA, 432 bp, Homo sapiens
cgctccccagtagaggacccggaaccagaactggaatccgcccttaccgcttgctgccaaaacagtgggggctgaactgacctctcccctttgggagagaaaaactgtctgggagcttgacaaaggcatgcaggagagaacaggagcagccacagccaggagggagagccttccccaagcaaacaatccagagcagctgtgcaaacaacggtgcataaatgaggcctcctggaccatgaagcgagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtg

SEQ ID NO: 9 - DNA, 22 bp, Artificial sequence
  TFF3 antisense oligonucleotide
tccttggctggcacggcacact

SEQ ID NO: 10 - DNA, 23 bp, Artificial sequence
  TFF3 antisense oligonucleotide
cgggagcaaagggacagaaaagc

SEQ ID NO: 11 - DNA, 23 bp, Artificial sequence
  TFF3 antisense oligonucleotide
gaagaactgtcctcgggtggagc

SEQ ID NO: 12 - DNA, 25 bp, Artificial sequence
  TFF3 antisense oligonucleotide
tcagaaagtctcaggcacgaagaac

SEQ ID NO: 13 - DNA, 25 bp, Artificial sequence
  TFF3 antisense oligonucleotide
gcagcagaaataaagcacaacctca

SEQ ID NO: 14 - DNA, 25 bp, Artificial sequence
  TFF3 antisense oligonucleotide
aacagtagcgagagtggttgtgaaa

SEQ ID NO: 15 - DNA, 22 bp, Artificial sequence
  TFF3 antisense oligonucleotide
cggcacggcacactggtttgca

SEQ ID NO: 16 - DNA, 25 bp, Artificial sequence
  TFF3 antisense oligonucleotide
ggtgcattctgtcttcctagtcagg

SEQ ID NO: 17 - DNA, 25 bp, Artificial sequence
  TFF3 antisense oligonucleotide
ggctccagatatgaactttcagcag

SEQ ID NO: 18 - DNA, 25 bp, Artificial sequence
  TFF3 antisense oligonucleotide
ggtggagcatgggacctttattcgt

SEQ ID NO: 19 - DNA, 22 bp, Artificial sequence
  TFF3 antisense oligonucleotide
tggcacggcacactggtttgca

SEQ ID NO: 20 - protein, 8 aa, Artificial sequence
  chemically synthesized peptide
AVPAKDRV

SEQ ID NO: 21 - protein, 8 aa, Artificial sequence
  chemically synthesized peptide
VPAKDRVD

SEQ ID NO: 22 - protein, 9 aa, Artificial sequence
  chemically synthesized peptide
AVPAKDRVD

SEQ ID NO: 23 - protein, 8 aa, Artificial sequence
  chemically synthesized peptide
GYPHVTPK

SEQ ID NO: 24 - protein, 8 aa, Artificial sequence
  chemically synthesized peptide
YPHVTPKE

SEQ ID NO: 25 - protein, 9 aa, Artificial sequence
  chemically synthesized peptide
GYPHVTPKE

SEQ ID NO: 26 - protein, 8 aa, Artificial sequence
  chemically synthesized peptide
FKPLQEAE

SEQ ID NO: 27 - protein, 8 aa, Artificial sequence
  chemically synthesized peptide
KPLQEAEC

SEQ ID NO: 28 - protein, 9 aa, Artificial sequence
  chemically synthesized peptide
FKPLQEAEC

(19) United States
(12) Patent Application Publication
(10) Pub. No.: US 2005/0186212 A1
(43) Pub. Date: Aug. 25, 2005

(54) Trefoil factor 3 (TFF3) as a target for anti-cancer therapy

(76) Inventors: Mary J. Janatpour, Castro Valley, CA (US); Pablo Garcia, Oakland, CA (US); Christoph Reinhard, Alameda, CA (US)

Correspondence Address: Chiron Corporation;Intellectual Property - R440, P.O. Box 8097, Emeryville, CA, 94662-8097, US

(21) Appl. No.: 10/913,685

(22) Filed: Aug. 5, 2004

Related U.S. Application Data
(60) Provisional application No. 60/493,173, filed on Aug. 7, 2003.
(60) Provisional application No. 60/498,438, filed on Aug. 28, 2003.

(51) Int. Cl. A61K 48/00; A61K 39/395

(52) U.S. Cl. 424/155.1; 514/44

(57) ABSTRACT

The present invention is directed to methods of treating and preventing cancer, such as cancer characterized by differential expression of trefoil factor 3 (TFF3). The methods include administering to a patient an agent that modulates TFF3 activity or expression. The present invention is further directed to reducing the physiological effects of TFF3 expression in cells, including inhibiting cell motility and resistance to apoptosis. Oligonucleotides and antibodies that can modulate expression or activity of TFF3 are also provided.

### CROSS REFERENCE TO RELATED APPLICATIONS

The present application claims priority benefit of U.S. Provisional Application 60/493,173, filed Aug. 7, 2003, and U.S. Provisional Application 60/498,438, filed Aug. 28, 2003, each of which is hereby incorporated by reference in its entirety.

### FIELD OF THE INVENTION

The present invention relates, in part, to agents that modulate activity or expression of trefoil factor 3 (TFF3). The present invention further relates to the use of these agents in treating, preventing, or detecting cancers.

### BACKGROUND OF THE INVENTION

Trefoil factor 3 (TFF3; or intestinal trefoil factor (ITF); or human intestinal trefoil factor (HITF)) is a protease-resistant peptide normally produced in intestinal goblet cells and secreted into the intestinal lumen (Thim, et al., Biochemistry, 1995, 34, 4757). TFF3 plays a role in epithelial restitution after injury (Mashimo, et al., Science, 1996, 274, 262 and Wong, et al., Biochemistry, 1995, 34, 4757) and is believed to accomplish this through several mechanisms: 1) protecting epithelial cells from apoptosis (Taupin, et al., Proc. Natl. Acad. Sci. USA, 2000, 97, 799); 2) inducing bordering cells to migrate and cover the wound (Dignass, et al., J. Clin. Invest., 1994, 94, 376); and 3) blocking deposition of serum complement system components that gain access to epithelial cells through the wound (Andoh, et al., J. Immunol., 2001, 167, 3887). Knockout mice are phenotypically normal unless their gut is wounded, in which case they die for failure to heal the wound. Recombinant TFF3 has been proposed for use in therapy related to irritable bowel syndrome and other bowel diseases (see, e.g., U.S. Pat. Nos. 6,063,755, 6,316,218 and U.S. Patent App. Publication No. 20010052483). Currently, TFF3 receptors are unknown.

The physiological effects of TFF3 in intestinal cells can be considered beneficial to cancer cell growth, proliferation, and metastasis. For example, resistance to apoptosis, induction of migration, and protection from complement activation are believed to be mechanisms through which cancer cells escape growth control, invade surrounding normal tissues, and metastasize or escape immune system surveillance. Clinical follow-up data have suggested that expression of TFF3 can be correlated with poor prognosis in gastric cancer (Yamachika, et al., Clin. Cancer Res., 2002, 8, 1092).

Other proteins, and the nucleic acids that encode them, have been reported to be useful as diagnostic and therapeutic targets for cancers (see, e.g., U.S. Pat. Nos. 6,261,562, 6,337,195, 6,465,611, 6,476,207, and U.S. Pat. App. Pub. No.: 20020192699). The compositions and methods described herein help meet current needs for new and more effective treatments for cancer and related diseases.

### SUMMARY OF THE INVENTION

The present invention provides methods of treating or preventing cancer comprising administering to a mammal afflicted with or predisposed to the cancer a therapeutically effective amount of a TFF3 neutralizing agent, wherein the TFF3 neutralizing agent is an antisense molecule, RNAi molecule, ribozyme, or small molecule.

In some embodiments the cancer is breast, colon, prostate, ovarian, or gastric cancer. In some embodiments TFF3 is differentially expressed in cells of said cancer.

In some embodiments the neutralizing agent is an antisense molecule comprising or overlaping a sequence corresponding to any of SEQ ID NOS: 5-19. In some embodiments the neutralizing agent is an RNAi molecule comprising or overlaping a sequence corresponding to any of SEQ ID NOS: 5-19. In some embodiments the neutralizing agent is an antibody which specifically binds to TFF3.

The present invention also provides methods of treating cancer comprising administering to a mammal afflicted with cancer or predisposed to cancer a therapeutically effective amount of a TFF3 neutralizing agent and providing the mammal with a traditional cancer treatment. In some embodiments the traditional cancer treatment is chemotherapy. In some embodiments the traditional cancer treatment is hormone ablation therapy. In some embodiments the hormone is an androgen.

The present invention further provides methods of inducing apoptosis in a cell comprising contacting the cell with a TFF3 neutralizing agent. In some embodiments the cell is mammalian. In some embodiments the cell is cancerous. In some embodiments the cell is a breast, prostate, colon, ovarian, or gastric cell.

In further embodiments the present invention provides methods of reducing tumor volume, slowing tumor growth, or preventing tumor growth comprising contacting the tumor with a TFF3 neutralizing agent. In some embodiments the tumor comprises cells in which TFF3 is differentially expressed.

The present invention provides methods of modulating at least one physiological effect associated with expression of TFF3 in a cell comprising contacting said cell with a TFF3 neutralizing agent. In some embodiments the physiological effect is increased cell motility or resistance to apoptosis.

The present invention also provides methods of inhibiting the migration, adhesion, or proliferation of a cell comprising contacting said cell with a TFF3 neutralizing agent.

In some embodiments the present invention provides methods of reducing invasiveness of a cancer cell comprising contacting the cancer cell with a TFF3 neutralizing agent.

The present invention further provides methods of modulating TFF3 expression in a cell comprising contacting the cell with a TFF3 neutralizing agent.

In further embodiments the present invention provides methods of detecting TFF3 in a biological sample comprising contacting the sample with a TFF3 neutralizing agent and detecting binding between the neutralizing agent and TFF3 in the sample. In some embodiments the TFF3 neutralizing agent comprises a detectable label.

Also, the present invention provides methods for detecting the presence of cancer in a biological sample comprising contacting the biological sample with a TFF3 neutralizing agent and detecting evidence of differential expression of TFF3 in the biological sample. Evidence of differential expression of TFF3 is indicative of the presence of cancer.

In some embodiments the “detecting” comprises comparing the results of the contacting with a control. In some embodiments the TFF3 neutralizing agent comprises a detectable label.

The present invention also provides methods for determining the susceptibility of a patient to a TFF3 neutralizing agent comprising detecting evidence of differential expression of TFF3 in the patient's cancer sample. Evidence of differential expression of TFF3 is indicative of the patient's susceptibility to the TFF3 neutralizing agent. In some embodiments the evidence of differential expression of TFF3 is upregulation of TFF3 in the patient's cancer sample. In some embodiments the patient's cancer sample is from breast, prostate, colon, ovarian, or gastric tissue.

In some embodiments the present invention provides methods for assessing the progression of cancer in a patient comprising comparing the expression of TFF3 in the patient at a first time point to the expression of TFF3 at a second time point. Increased expression of TFF3 at the second time point relative to the first time point is indicative of progression of the cancer. In some embodiments the “increased expression of TFF3” is increased expression of at least about 25%, at least about 50%, at least about 75%, or at least about 90%.

The present invention also provides methods for detecting an increased risk of metastasis of a cancer in a patient comprising comparing the expression of TFF3 in the patient at a first time point to the expression of TFF3 at a second time point. Increased expression of TFF3 at the second time point relative to the first time point is indicative of an increased risk of metastasis.

The present invention also provides antisense molecules that modulate expression of TFF3. In some embodiments the antisense molecule comprises or overlaps a sequence of SEQ ID NOS: 5-19. In some embodiments the antisense molecules comprise any of SEQ ID NOS: 5-19.

The present invention also provides RNAi molecules that modulate expression of TFF3. In some embodiments the RNAi molecule comprises or overlaps a sequence of SEQ ID NOS: 5-19. In some embodiments the RNAi molecules comprise any of SEQ ID NOS: 5-19.

The present invention also provides compositions comprising antisense molecules and/or RNAi molecules and a pharmaceutically acceptable carrier.

The present invention also provides isolated anti-TFF3 antibodies, wherein the antibodies recognize at least one region of TFF3 sequence corresponding to SEQ ID NO: 20, 21, 22, 23, 24, 25, 26, 27 or 28. In some embodiments the antibody is produced by a process comprising:
a) synthesizing a library of antibodies on phage;
b) panning the library against a sample by bringing the phage into contact with a composition comprising at least one region of TFF3 sequence corresponding to SEQ ID NO: 20, 21, 22, 23, 24, 25, 26, 27 or 28;
c) isolating phage which bind the composition, wherein the antibody is characterized by its ability to bind to at least one region of TFF3 sequence corresponding to SEQ ID NO: 20, 21, 22, 23, 24, 25, 26, 27 or 28 with a binding affinity of at least 10⁸ l/; and
d) analyzing the isolated phage to determine a sequence encoding an amino acid sequence to which the at least one region of TFF3 sequence corresponding to SEQ ID NO: 20, 21, 22, 23, 24, 25, 26, 27 or 28 binds.

In some embodiments the antibody is a monoclonal antibody, a polyclonal antibody, a chimeric antibody, a humanized antibody, a single-chain antibody or a Fab fragment. In some embodiments the antibody is labeled. In some embodiments the label is an enzyme, radioisotope, or fluorophore. In some embodiments the binding affinity of the antibody is less than about 1×10⁵ Kₐ for a polypeptide other than TFF3.

The present invention further provides isolated cells and hybridomas that produce the antibodies of the present invention.

The present invention also provides isolated polypeptides comprising three or fewer amino acid sequences selected from SEQ ID NOS: 13, 20, 21, 22, 23, 24, 25, 26, 27 or 28. In some embodiments the polypeptide is from about 8 to about 80 amino acids in length. In some embodiments the polypeptide binds specifically to an anti-TFF3 antibody.

The present invention further provides methods of using an antibody to detect differential expression of TFF3 in a sample comprising combining the antibodies of the present invention with said sample under conditions which allow the formation of antibody:TFF3 complexes; measuring the amount of complexes; and comparing the amount of complexes to a control. Elevated levels of complex in the sample indicates differential expression of TFF3.

The present invention also provides isolated epitope-bearing fragments of the polypeptide of SEQ ID NOS: 1-4. In some embodiments the fragments comprises between about 6 and about 20 contiguous amino acids of SEQ ID NO:1-4. In some embodiments the fragment comprises about 10 contiguous amino acids of SEQ ID NO: 1-4. In some embodiments the polypeptide is SEQ ID NO:2. In some embodiments the fragment comprises SEQ ID NOS: 20, 21, 22, 23, 24, 25, 26, 27 or 28.

Also, the present invention provides isolated anti-TFF3 antibodies obtained by immunization of a subject with the epitope-bearing fragment of the present invention. In some embodiments the present invention provides the isolated antibodies in pharmaceutical compositions with one or more pharmaceutically acceptable carrier. In some embodiments the antibody neutralizes TFF3.

The present invention also provides methods for generating an antibody for the treatment of cancer comprising identifying an antibody that binds to and neutralizes TFF3, and expressing the antibody in a recombinant expression host cell.

Further, the present invention provides pharmaceutical compositions comprising an antibody that binds to and neutralizes TFF3, and a pharmaceutically acceptable carrier, wherein the antibody was generated using a recombinant host cell. In some embodiments the recombinant host cell is selected from the group consisting of Chinese Hamster Ovary cell, myeloma cell and bacterial host cell.

### BRIEF DESCRIPTION OF THE DRAWINGS

FIG. 1 depicts variation of TFF3 mRNA expression in different whole tissues.

FIG. 2 depicts variation of expression levels of TFF3 in different cell lines.

FIG. 3 depicts varying effectivenes of different TFF3 antisense oligonucleotides in knocking down TFF3 mRNA expression levels in colon cancer cells.

FIG. 4 depicts specificity of TFF3 antisense oligonucleotides.

FIG. 5 depicts specificity of a further TFF3 antisense oligonucleotides.

FIG. 6 depicts the effectiveness of antisense oligonucleotides in knocking down TFF3 mRNA expression in a cancer cell line.

FIG. 7 depicts cytotoxic and anti-proliferative effects of antisense oligonucleotides in colon cancer cells.

FIGS. 8A and 8B depict cytotoxic and anti-proliferative effects of antisense oligonucleotides in prostate cancer cells.

FIG. 9 depicts the non-toxicity of TFF3 antisense oligonucleotides in normal cells.

FIG. 10 depicts TFF3 antisense oligonucleotide inhibition of colony growth in soft agar media for prostate cancer cells.

FIG. 11 depicts inhibition of cell proliferation using anti-TFF3 antibodies. IgG fractions isolated from the anti-TFF3 polyclonal antisera were diluted to a final concentration of 50 μg/ml in growth medium containing 1% FBS, and were then added to quadruplicate wells on Days 0 and 4 of the proliferation assay. The degree of cell proliferation was scored on Day 7.

### DETAILED DESCRIPTION OF EMBODIMENTS

The present invention provides, inter alia, TFF3 neutralizing agents and methods of preventing, treating, and detecting cancer using these agents. TFF3 polypeptides can be differentially expressed (e.g., at the protein or mRNA level) in many cancer tissues and cancer cell lines, such as in connection with prostate cancer, breast cancer, ovarian cancer, gastric cancer, and colon cancer. As exemplified herein, knockdown of TFF3 expression (such as by using oligonucleotide-based techniques) can lead to cytotoxicity and inhibition of anchorage-independent growth. Accordingly, inhibition of TFF3 polypeptide activity or knockdown of TFF3 polypeptide or polynucleotide expression in cells using appropriate neutralizing agents can help prevent or treat cancers characterized, for example, by expression of TFF3. In some embodiments, the cancer tissue can differentially express (e.g., show upregulation or downregulation of) TFF3.

As used herein, the phrase “differentially expressed” generally refers to a polypeptide or polynucleotide that is expressed, at higher or lower levels in cancer cells, e.g., mRNA is found at levels at least about 25%, at least about 50% to about 75%, at least about 90%, at least about 95%, at least about 1.5-fold, at least about 2-fold, at least about 5-fold, at least about 10-fold, or at least about 50-fold or more, different (e.g.,, higher or lower) in a cancer cell when compared with a cell of the same cell type that is not cancerous (normal cell). The comparison can be made between two tissues, for example, if one is using in situ hybridization or another assay method that allows some degree of discrimination among cell types in the tissue. The comparison may also be made between cells removed from their tissue source. TFF3 is a gene that has been shown to be differentially expressed in colon, breast, prostate and other cancers. In some embodiments, differential expression is observed as higher than normal expression. Methods for determining polypeptides and polynucleotides that are differentially expressed in certain cells are further described in U.S. Pat. App. Pub. No.: 20020192699 and U.S. Pat. No. 6,476,207. Gene products that are differentially expressed in cancerous prostate cells are described in U.S. Ser. No. 10/310,673.

As used herein, the term “about” refers to +/−10% of a value.

TFF3 Polypeptides

“TFF3” or “TFF3 polypeptides,” as used herein, refer to proteins (polypeptides) or fragments thereof that are substantially homologous to human trefoil factor 3 (SEQ ID NOS: 1-4, GenBank gi:4507453, Q07654, NP_{—}003217, AAH17859, BAA95531, BAB13731). The polypeptides contemplated by the invention include those encoded by the disclosed TFF3 polynucleotides (SEQ ID NOS: 5-8, GenBank gi:4507452, BC017859, AF432265), as well as nucleic acids that, by virtue of the degeneracy of the genetic code, are not identical in sequence to the disclosed TFF3 polynucleotides. Thus, the invention includes within its scope a polypeptide encoded by a polynucleotide having the sequence of any one of the polynucleotide sequences provided herein, or a variant thereof. In some embodiments TFF3 comprises an amino acid sequence of SEQ ID NOS: 1-4. In some preferred embodiments, TFF3 comprises an amino acid sequence of SEQ ID NO:2.

The terms “polypeptide” and “protein,” are used interchangeably and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. The term also includes “peptide” which is a polypeptide that is from 2 to about 30 amino acids in length. Polypeptides can be refer to both a full length polypeptide encoded by a recited polynucleotide (such as a TFF3 polynucleotide), the polypeptide encoded by the gene represented by the recited polynucleotide, as well as portions or fragments thereof.

“Polypeptides” also include variants of the naturally occurring proteins, where such variants are homologous or substantially similar to the naturally occurring protein, and can be of an origin of the same or different species as the naturally occurring protein (e.g., human, murine, or some other species that naturally expresses the recited polypeptide, usually a mammalian species). In general, variant TFF3 polypeptides have a sequence that has at least about 80%, usually at least about 90%, and more usually at least about 95% sequence identity or higher, i.e. 96%, 97%, 98% or 99% sequence identity with a differentially expressed polypeptide described herein, as can be measured by BLAST using, for example, default parameters. The variant polypeptides can be naturally or non-naturally glycosylated, i.e., the polypeptide has a glycosylation pattern that differs from the glycosylation pattern found in the corresponding naturally occurring protein.

The invention also encompasses homologs of TFF3 polypeptides (or fragments thereof) where the homologs are isolated from other species naturally occurring glycosylated TFF3 include those produced by normal and neoplastic cells, which may exhibit different glycosylation patterns, i.e. other animal or plant species, where such homologs, usually mammalian species, e.g. rodents, such as mice, rats; domestic animals, e.g., horse, cow, dog, cat; and humans. By “homolog” is meant a polypeptide having at least about 35%, usually at least about 40% and more usually at least about 60% amino acid sequence identity to a particular protein as identified above, where sequence identity is determined using the BLAST algorithm, with, for example, default parameters.

In general, the TFF3 polypeptides of the subject invention are provided in a non-naturally occurring environment, e.g. are separated from their naturally occurring environment. In certain embodiments, the subject protein is present in a composition that is enriched for the protein as compared to a control. As such, purified polypeptide is provided, whereby “purified” is meant that the protein is present in a composition that is substantially free of other polypeptides, whereby “substantially free” is meant that less than about 90%, usually less than about 60%, or more usually less than 50% of the composition is made up of other polypeptides.

Also within the scope of the invention are variants; variants of polypeptides include mutants, fragments, and fusions. Mutants can include amino acid substitutions, additions or deletions. The amino acid substitutions can be conservative amino acid substitutions or substitutions to eliminate non-essential amino acids, such as to alter a glycosylation site, a phosphorylation site or an acetylation site, or to minimize misfolding by substitution or deletion of one or more cysteine residues that are not necessary for function. Conservative amino acid substitutions are those that preserve the general charge, hydrophobicity/hydrophilicity, and/or steric bulk of the amino acid substituted. Variants can be designed so as to retain or have enhanced biological activity of a particular region of the protein (e.g., a functional domain and/or, where the polypeptide is a member of a protein family, a region associated with a consensus sequence). Selection of amino acid alterations for production of variants can be based upon the accessibility (interior vs. exterior) of the amino acid (see, e.g., Go et al, Int. J. Peptide Protein Res. (1980) 15:211), the thermostability of the variant polypeptide (see, e.g., Querol et al., Prot. Eng. (1996) 9:265 which is incorporated herein by reference in its entirety), desired glycosylation sites (see, e.g., Olsen and Thomsen, J. Gen. Microbiol. (1991) 137:579), desired disulfide bridges (see, e.g., Clarke et al., Biochemistry (1993) 32:4322; and Wakarchuk et al., Protein Eng. (1994) 7:1379), desired metal binding sites (see, e.g., Toma et al., Biochemistry (1991) 30:97, and Haezerbrouck et al., Protein Eng. (1993) 6:643), and desired substitutions with in proline loops (see, e.g., Masul et al., Appl. Env. Microbiol. (1994) 60:3579 which is incorporated herein by reference in its entirety). Cysteine-depleted muteins can be produced as disclosed in U.S. Pat. No. 4,959,314 which in incorporated herein by reference in its entirety.

Variants can also include fragments of the polypeptides disclosed herein, particularly biologically active fragments and/or fragments corresponding to functional domains. Fragments of interest will typically be at least about 10 aa to at least about 15 aa in length, at least about 50 aa in length, and can be as long as 300 aa in length or longer, and in some embodiments do not exceed about 1000 aa in length, where the fragment will have a stretch of amino acids that is identical to a polypeptide encoded by a polynucleotide having a sequence of any one of the polynucleotide sequences provided herein, or a homolog thereof. The protein variants described herein are encoded by polynucleotides that are within the scope of the invention. The genetic code can be used to select the appropriate codons to construct the corresponding variants. In particular, fragments will include those that contain the specific domains or epitopes of the TFF3 protein.

TFF3 Polynucleotides

In some embodiments, the present invention relates to the inhibition or detection of a TFF3 polynucleotide encoding TFF3 that is differentially expressed in some cancers, such as for example, prostate, breast, ovarian, or colon cancer. In some embodiments TFF3 is encoded for by a nucleic acid having a nucleotide sequence of SEQ ID NOS: 5-8. In some preferred embodiments, TFF3 is encoded for by a nucleic acid having a nucleotide sequence of SEQ ID NO: 7.

The scope of the invention with respect to polynucleotide compositions useful in the methods described herein includes, but is not limited to, polynucleotides having a sequence set forth in any one of the polynucleotide sequences provided herein (e.g., SEQ ID NO:2); polynucleotides obtained from the biological materials described herein or other biological sources (particularly human sources) by hybridization under stringent conditions (particularly conditions of high stringency); genes corresponding to the provided polynucleotides; variants of the provided polynucleotides and their corresponding genes, particularly those variants that retain a biological activity of the encoded gene product (e.g., a biological activity ascribed to a gene product corresponding to the provided polynucleotides as a result of the assignment of the gene product to a protein family(ies) and/or identification of a functional domain present in the gene product). Other nucleic acid compositions contemplated by and within the scope of the present invention will be readily apparent to one of ordinary skill in the art when provided with the disclosure here. “Polynucleotide” and “nucleic acid” as used herein with reference to nucleic acids of the composition is not intended to be limiting as to the length or structure of the nucleic acid unless specifically indicted.

TFF3 polynucleotides can be expressed in human cancer tissues, such as human prostate, breast, and colon tissue. Nucleic acid compositions described herein of particular interest comprise a sequence set forth in any one of the polynucleotide sequences provided herein or an identifying sequence thereof. An “identifying sequence” is a contiguous sequence of residues at least about 10 nt to about 20 nt in length, usually at least about 50 nt to about 100 nt in length, that uniquely identifies a polynucleotide sequence, e.g., exhibits less than 90%, usually less than about 80% to about 85% sequence identity to any contiguous nucleotide sequence of more than about 20 nt. Thus, the subject nucleic acid compositions include full length cDNAs or mRNAs that encompass an identifying sequence of contiguous nucleotides from any one of the polynucleotide sequences provided herein.

The polynucleotides useful in the methods described herein also include polynucleotides having sequence similarity or sequence identity with native TFF3 DNA. This includes associated 5′ and 3′ untranslated sequences, promoter and enhancer sequences and sequences in sense or antisense orientation. Nucleic acids having sequence similarity are detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under stringent conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences under stringent hybridization conditions. By using probes, particularly labeled probes of DNA sequences, one can isolate homologous or related genes. The source of homologous genes can be any species, e.g. primate species, particularly human; rodents, such as rats and mice; canines, felines, bovines, ovines, equines, yeast, nematodes, etc.

In one embodiment, hybridization is performed using at least 15 contiguous nucleotides (nt) of at least one of the polynucleotide sequences provided herein. That is, when at least 15 contiguous nt of one of the disclosed polynucleotide sequences are used as a probe, the probe will preferentially hybridize with a nucleic acid comprising the complementary sequence, allowing the identification and retrieval of the nucleic acids that uniquely hybridize to the selected probe. Probes from more than one polynucleotide sequences provided herein can hybridize with the same nucleic acid if the cDNA from which they were derived corresponds to one mRNA. Probes of more than 15 nt can be used, e.g., probes of a size within the range of about 18 nt, 25 nt, 50 nt, 75 nt or 100 nt, but in general about 15 nt represents sufficient sequence for unique identification.

Polynucleotides contemplated by the invention also include naturally occurring variants of the nucleotide sequences (e.g., degenerate variants, allelic variants, etc.). Variants of the polynucleotides contemplated by the invention are identified by hybridization of putative variants with nucleotide sequences disclosed herein, preferably by hybridization under stringent conditions. For example, by using appropriate wash conditions, variants of the polynucleotides described herein can be identified where the allelic variant exhibits at most about 25-30% base pair (bp) mismatches relative to the selected polynucleotide probe. In general, allelic variants contain about 15 to about 25% bp mismatches, and can contain as little as even about 5-15%, or about 2-5%, or about I-2% bp mismatches, as well as a single bp mismatch.

The invention also encompasses homologs corresponding to the TFF3 polynucleotide sequences provided herein, where the source of homologous genes can be any mammalian species, e.g., primate species, particularly human; rodents, such as rats; canines, felines, bovines, ovines, equines, yeast, nematodes, etc. Between mammalian species, e.g., human and mouse, homologs generally have substantial sequence similarity to a TFF3 gene or portion thereof, such as, for example, at least 75% sequence identity, at least 90%, at least 95%, 96%, 97%, 98% or 99% between nucleotide sequences. Sequence similarity is calculated based on a reference sequence, which may be a subset of a larger sequence, preferably the extracellular coding sequence, e.g. as a conserved motif, part of coding region, flanking region, etc. A reference sequence will usually be at least about 18 contiguous nt long, more usually at least about 30 nt long, and may extend to the complete sequence that is being compared. Algorithms for sequence analysis are known in the art, such as gapped BLAST using default parameters, described in Altschul, et al. Nucleic Acids Res. (1997) 25:3389-3402.

In general, variants of the TFF3 polynucleotides described herein have a sequence identity greater than at least about 65%, preferably at least about 75%, more preferably at least about 85%, and can be greater than at least about 90%, 95%, 96%, 98%, 99% or more as determined by the Smith-Waterman homology search algorithm as implemented in MPSRCH program (Oxford Molecular). An example method of calculating percent identity is the Smith-Waterman algorithm, using the following: global DNA sequence identity greater than 65% as determined by the Smith-Waterman homology search algorithm as implemented in MPSRCH program (Oxford Molecular) using an affine gap search with the following search parameters: gap open penalty, 12; and gap extension penalty, 1.

The subject nucleic acids can be cDNAs or genomic DNAs, as well as fragments thereof, particularly fragments that encode a biologically active gene product and/or are useful in the methods disclosed herein (e.g., in diagnosis, as a unique identifier of a differentially expressed gene of interest, etc.). The term “cDNA” as used herein is intended to include all nucleic acids that share the arrangement of sequence elements found in native mature mRNA species, where sequence elements are exons and 3′ and 5′ non-coding regions. Normally mRNA species have contiguous exons, with the intervening introns, when present, being removed by nuclear RNA splicing, to create a continuous open reading frame encoding a polypeptide. mRNA species can also exist with both exons and introns, where the introns may be removed by alternative splicing. Furthermore it should be noted that different species of mRNAs encoded by the same genomic sequence can exist at varying levels in a cell, and detection of these various levels of mRNA species can be indicative of differential expression of the encoded gene product in the cell.

A genomic sequence of interest comprises the nucleic acid present between the initiation codon and the stop codon, as defined in the listed sequences, including all of the introns that are normally present in a native chromosome. It can further include the 3′ and 5′ untranslated regions found in the mature mRNA. It can further include specific transcriptional and translational regulatory sequences, such as promoters, enhancers, etc., including about 1 kb, but possibly more, of flanking genomic DNA at either the 5′ and 3′ end of the transcribed region. The genomic DNA can be isolated as a fragment of 100 kbp or smaller; and substantially free of flanking chromosomal sequence. The genomic DNA flanking the coding region, either 3′ and 5′, or internal regulatory sequences as sometimes found in introns, contains sequences required for proper tissue, stage-specific, or disease-state specific expression.

The nucleic acids of the subject invention can encode all or a part of the subject TFF3 polypeptides or may comprise non-coding sequences, e.g. from the 5′ or 3′ non-coding region of the gene. As noted, these DNAs or RNAs may be in the sense or antisense orientation. Double or single stranded fragments can be obtained from the DNA sequence by chemically synthesizing oligonucleotides in accordance with conventional methods, by restriction enzyme digestion, by PCR amplification, etc. Isolated polynucleotides and polynucleotide fragments contemplated by the invention comprise at least about 10, about 15, about 20, about 35, about 50, about 100, about 150 to about 200, about 250 to about 300, or about 350 contiguous nt selected from the polynucleotide provided herein. For the most part, fragments will be of at least 15 nt, usually at least 18 nt or 25 nt, and up to at least about 50 contiguous nt in length or more. In some embodiments, the polynucleotide molecules comprise a contiguous sequence of at least 12 nt selected from any one of the polynucleotide sequences provided herein.

Oligonucleotide probes specific to the TFF3 polynucleotides can be generated using the TFF3 polynucleotide sequences disclosed herein. The probes are preferably at least about 12 nt, 15 nt, 16 nt, 18 nt, 20 nt, 22 nt, 24 nt, or 25 nt fragments of a corresponding contiguous sequence of any one of the polynucleotide sequences provided herein, and can be less than 2 kb, 1 kb, 0.5 kb, 0.1 kb, or 0.05 kb in length. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of any one of the polynucleotide sequences provided herein. More preferably, probes are designed based on a contiguous sequence of one of the subject polynucleotides that remain unmasked following application of a masking program for masking low complexity (e.g., XBLAST) to the sequence, i.e., one would select an unmasked region, as indicated by the polynucleotides outside the poly-n stretches of the masked sequence produced by the masking program.

The TFF3 polynucleotides of the subject invention can be isolated and obtained in substantial purity, generally in a form other than an intact chromosome. Typically, the polynucleotides, either as DNA or RNA, can be obtained substantially free of other naturally-occurring nucleic acid sequences, generally being at least about 50%, usually at least about 90% pure and are typically “recombinant”, e.g., flanked by one or more nucleotides with which it is not normally associated on a naturally occurring chromosome.

TFF3 polynucleotides described herein can be provided as a linear molecule or within a circular molecule, and can be provided within autonomously replicating molecules (vectors) or within molecules without replication sequences. Expression of the polynucleotides can be regulated by their own or by other regulatory sequences known in the art. The polynucleotides can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

The nucleic acid compositions described herein can be used to, for example, produce polypeptides, (which may be used to obtain anti-TFF3 antibodies) as probes for the detection of mRNA in biological samples (e.g., extracts of human cells) to generate additional copies of the polynucleotides, to generate ribozymes or antisense oligonucleotides, and as single stranded DNA probes or as triple-strand forming oligonucleotides. The probes described herein can be used to, for example, determine the presence or absence of any one of the polynucleotides provided herein or variants thereof in a sample. These and other uses are described in further detail below.

The term “overlap,” as used herein, refers to the region of sequence shared by more than one polynucleotides or oligonucleotides. For example, an oligonucleotide that overlaps a further oligonucleotide has a region of sequence that substantially corresponds to a region of sequence in the further oligonucleotide. In some embodiments, the overlap can be at least about 3, at least about 4, at least about 5, at least about 6, at least about 7, at least about 8, at least about 9, at least about 10, at least about 15, at least about 20, at least about 50, at least about 100, or more nucleotides in length.

Neutralizing Agents

The methods and articles of manufacture of the present invention use, or incorporate, a TFF3 neutralizing agent that modulates TFF3 activity or expression in cells. As used herein, the term “modulating” refers to a change in the quality or quantity of a gene, protein, or any molecule that is inside, outside, or on the surface of a cell. The change can be an increase or decrease in expression or level of a molecule. The term “modulates” also includes changing the quality or quantity of a biological function/activity such as, for example, proliferation, secretion, adhesion, apoptosis, cell-to-cell signaling, and the like. In some embodiments, modulation of activity or expression can be more than about 10%, more than about 20%, more than about 30%, more than about 40%, or more than about 50%.

A variety of neutralizing agents are contemplated herein, such as small molecules, peptides, antibodies, antisense molecules, ribozymes, RNAi molecules, and the like.

Small Molecules

The neutralizing agent can comprise a small molecule optionally fused to, or conjugated with, a cytotoxic agent (such as those described herein). Libraries of small molecules can be screened against TFF3 or TFF3-expressing cells in order to identify a small molecule which binds to that antigen. The small molecule may further be screened for its antagonistic or neutralizing properties and/or conjugated with a cytotoxic agent according to well known methods.

Peptides

The neutralizing agent can also be a peptide generated by rational design or by phage display (see, e.g., WO98/35036). In one embodiment, the molecule of choice may be a “CDR mimic” or antibody analogue designed based on the CDRs of an antibody. While such peptides may be antagonistic by themselves, the peptide may optionally be fused to a cytotoxic agent so as to add or enhance antagonistic properties of the peptide. Peptides can comprise 2 to about 20, 2 to about 15, or 2 to about 10 amino acids.

Antisense Oligonucleotides

In certain circumstances, it may be desirable to modulate (e.g., decrease) the amount of TFF3 expressed in a cell. Thus, in another aspect of the present invention, TFF3 anti-sense oligonucleotides can be made and a method utilized for diminishing the level of expression of TFF3 by a cell comprising administering one or more TFF3 anti-sense oligonucleotides. By the phrase “TFF3 antisense oligonucleotides,” reference is made to oligonucleotides that have a nucleotide sequence that interacts through base pairing with a complementary nucleic acid sequence involved in the expression of TFF3 such that the expression of TFF3 is reduced. Preferably, the specific nucleic acid sequence involved in the expression of TFF3 is a genomic DNA molecule or mRNA molecule that encodes TFF3. This genomic DNA molecule can comprise regulatory regions of the TFF3 gene, and/or the coding sequence for mature TFF3 protein.

The term “complementary” in the context of TFF3 antisense oligonucleotides and methods therefor means sufficiently complementary to such a sequence as to allow hybridization to that sequence in a cell, i.e., under physiological conditions. The TFF3 antisense oligonucleotides preferably comprise a sequence containing from about 8 to about 100 nucleotides and more preferably the antisense oligonucleotides comprise from about 15 to about 30 nucleotides or about 18 to about 26 nucleotides.

The TFF3 antisense oligonucleotides can also contain a variety of modifications that confer resistance to nucleolytic degradation such as, for example, modified internucleoside linkages [Uhlmann and Peyman, Chemical Reviews 90:543-548 (1990); Schneider and Banner, Tetrahedron Lett. 31:335, (1990)], modified nucleic acid bases as disclosed in 5,958,773, and patents disclosed therein, and/or sugars and the like. Further description of modified oligonucleotides is provided infra.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773. Further description of modified oligonucleotides is provided infra.

The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. Further description of modified bases and antisense oligonucleotide conjugates is provided infra.

In the antisense art, a certain degree of routine experimentation can be carried out to select optimal antisense molecules for particular targets. In its design, the antisense molecule can be targeted to an accessible, or exposed, portion of the target RNA molecule. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. For example, RNA from treated and control cells can be reverse-transcribed and the resulting cDNA populations analyzed. (Branch, A. D., T.I.B.S. 23:45-50 (1998)).

Antisense molecules of the present invention include oligonucleotides that are complementary to regions or portions of a TFF3 polynucleotide, such as a nucleic acid having a nucleotide sequence of SEQ ID NOS: 5-8. Some example antisense oligonucleotides that can, alone or in combination, reduce expression of TTF3 in cells include those having the sequences provided in Table 1 below, or fragments thereof.

TABLE 1
Example TFF3 antisense oligonucleotides

| | |
|---|---|
| SEQ ID NO | Sequence |

| | |
|---|---|
| SEQ ID NO: 9 | 5′-TCCTTGGCTGGCACGGCACACT-3′ |
| SEQ ID NO: 10 | 5′-CGGGAGCAAAGGGACAGAAAAGC-3′ |
| SEQ ID NO: 11 | 5′-GAAGAACTGTCCTCGGGTGGAGC-3′ |
| SEQ ID NO: 12 | 5′-TCAGAAAGTCTCAGGCACGAAGAAC-3′ |
| SEQ ID NO: 13 | 5′-GCAGCAGAAATAAAGCACAACCTCA-3′ |
| SEQ ID NO: 14 | 5′-AACAGTAGCGAGAGTGGTTGTGAAA-3′ |
| SEQ ID NO: 15 | 5′-CGGCACGGCACACTGGTTTGCA-3′ |
| SEQ ID NO: 16 | 5′-GGTGCATTCTGTCTTCCTAGTCAGG-3′ |
| SEQ ID NO: 17 | 5′-GGCTCCAGATATGAACTTTCAGCAG-3′ |
| SEQ ID NO: 18 | 5′-GGTGGAGCATGGGACCTTTATTCGT-3′ |
| SEQ ID NO: 19 | 5′-TGGCACGGCACACTGGTTTGCA-3′ |

The specificity and sensitivity of antisense has been harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be useful in treatment regimes for treatment of cells, tissues and mammals, including humans.

Some examples of antisense compounds useful in this invention include oligonucleotides containing modified backbones or non-natural internucleoside linkages. Oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Example modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thiono-alkylphosphonates, thionoalkylphosphotriesters, and borano-phosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Some example modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In other oligonucleotide mimetics, both the sugar and the internucleoside linkage, i.e., the backbone, of the nucleotide units are replaced with novel groups. The base units can be maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In further embodiments of the invention are oligonucleotides with phosphorothioate backbones and oligonucleosides with heteroatom backbones, such as provided in U.S. Pat. No. 5,489,677 and U.S. Pat. No. 5,602,240. Also provided are oligonucleotides having morpholino backbone structures of the above-referenced U.S. Pat. No. 5,034,506.

Modified oligonucleotides may also contain one or more substituted sugar moieties. For example, oligonucleotides can comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941, and 5,750,692, each of which is herein incorporated by reference in its entirety.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications can be incorporated in a single compound or even at a single nucleoside within an oligonucleotide.

The present invention also includes antisense compounds that are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention can be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention can be synthesized in vitro. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention further encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the antisense compounds of the invention, pharmaceutically acceptable salts of such prodrugs, and other bioequivalents.

The antisense compounds of the present invention can be utilized for diagnostics, therapeutics, prophylaxis and as research reagents and kits. For therapeutics, an animal, preferably a human, suspected of having a disease or disorder which can be treated by modulating the expression of TFF3 is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay tumor formation or metastasis, for example.

The antisense compounds of the invention are useful for research and diagnostics, because these compounds hybridize to nucleic acids encoding TFF3, enabling sandwich and other assays to easily be constructed to exploit this fact. Hybridization of the antisense oligonucleotides of the invention with a nucleic acid encoding TFF3 can be detected by means known in the art. Such means may include conjugation of an enzyme to the oligonucleotide, radiolabelling of the oligonucleotide or any other suitable detection means. Kits using such detection means for detecting the level of TFF3 in a sample may also be prepared.

The present invention also includes pharmaceutical compositions and formulations that include the antisense compounds of the invention. In some embodiments, compositions containing antisense molecules can be prepared and formulated as emulsions. Emulsions are typically heterogenous systems of one liquid dispersed in another in the form of droplets usually exceeding 0.1 μm in diameter. Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

In another related embodiment, methods and compositions of the invention may contain one or more antisense molecules, particularly oligonucleotides, targeted to a first nucleic acid and one or more additional antisense compounds targeted to a second nucleic acid target. Two or more combined compounds may be used together or sequentially. In some embodiments, at least one of the targeted nucleic acids encodes TFF3.

Ribozymes

The neutralizing agent can also be a ribozyme that, for example, inhibits TFF3 expression. Ribozymes are typically catalytic RNA molecules with ribonucleic activity that are capable of clearing a single-stranded nucleic acid, such as an mRNA, to which they have a complementary region. Because a ribozyme is an enzyme, a single ribozyme molecule can cleave many molecules of target RNA. In addition, the ribozyme is typically a highly specific inhibitor, with the specificity of inhibition depending not only on the base pairing mechanism of binding to the target RNA, but also on the mechanism of target RNA cleavage. Ribozyme molecules designed to catalytically cleave target gene mRNA transcripts can also be used to prevent translation of target gene mRNA and, therefore, expression of target gene product. (See, e.g., WO 90/11364 and Sarver, et al., 1990, Science 247, 1222-1225).

Ribozymes can be designed to anneal to various sites in the target RNA. The binding arms can be complementary to the target site, such as a TFF3 polynucleotide. The ribozymes can be chemically synthesized. Example synthetic methods can follow the procedure for normal RNA synthesis as described in Usman et al., 1987 J. Am. Chem. Soc., 109, 7845-7854 and in Scaringe et al., 1990 Nucleic Acids Res., 18, 5433-5441, and can make use of common nucleic acid protecting and coupling groups, such as dimethoxytrityl at the 5′-end, and phosphoramidites at the 3′-end. The average stepwise coupling yields can be >98%. Hairpin ribozymes can also be synthesized in two parts and annealed to reconstruct the active ribozyme (Chowrira and Burke, 1992 Nucleic Acids Res., 20, 2835-2840). Ribozymes can be modified to enhance stability by modification with nuclease resistant groups, for example, 2′-amino, 2′-C-allyl, 2′-flouro, 2′-o-methyl, 2′-H (see, e.g., Usman and Gedergren, 1992 TIBS 17, 34, which is incorporated herein by reference).

Ribozymes can be purified by methods known in the art such as gel electrophoresis or by high pressure liquid chromatography (HPLC).

Ribozyme activity can be optimized by chemically synthesizing ribozymes with modifications that prevent their degradation by serum ribonucleases (see e.g., Eckstein et al., WO 92/07065; Perrault et al., Nature, 1990, 344:565; Pieken et al., Science 1991, 253:314; Usman and Cedergren, Trends in Biochem. Sci. 1992, 17:334; Usman et al., WO 93/15187; and Rossi et al., WO 91/03162, and U.S. Pat. No. 5,652,094, each of which is incorporated herein by reference, and which describe various chemical modifications that can be made to the sugar moieties of enzymatic RNA molecules.

Thus, ribozymes (e.g., hairpin, hammerhead, or RNAase P ribozyme, as well as a minizyme (McCall, M. (1992) Proc. Natl. Acad. Sci. USA 89:5710-5714), or other catalytic RNA molecule) can be used to catalytically clear TFF3 transcripts to thereby inhibit translation of TFF3 mRNA. A ribozyme having specificity for TFF3 can be designed based on the nucleotide sequence of TFF3, e.g., a human TFF3 DNA sequence provided herein or related polynucleotide. Techniques for synthesizing ribozymes are disclosed in Cech et al., U.S. Pat. Nos. 4,987,071 and 5,116,742 Alternatively, TFF3 mRNA can be used to select a catalytic RNA having a specific ribonucleic activity from a pool of RNA molecules. (See, e.g., Bartel and Stostak, J. W., J. Biol. Chem. 1261: 1411-1418(1993)).

Alternatively, TFF3 expression can be inhibited by targeting nucleotide sequences that are complementary to the regulating region of TFF3 (promoter, enhancer) to form triple helical structures that prevent transcription in target cells. (See, e.g., Helene et al., Annal. NY Acad. Sci. 660: 27-36 (1992)).

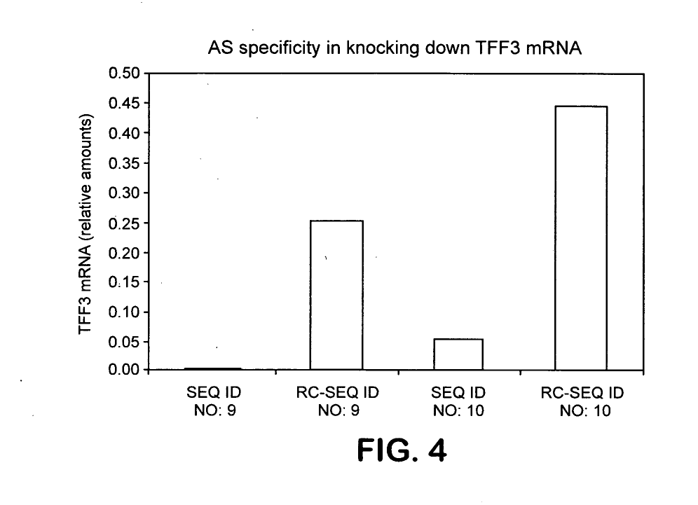

According to some embodiments, the neutralizing agent is a hammerhead ribozyme. Hammerhead ribozymes are believed to cleave mRNAs at locations dictated by flanking regions that form complementary base pairs with the target mRNA. A feature of using hammerhead ribozymes is that the target mRNA has the following sequence of two bases: 5′-UG-3′. The construction and production of hammerhead ribozymes is well known in the art and described in Myers, 1995, Molecular Biology and Biotechnology: A Comprehensive Desk Reference, VCH Publishers, New York, (see especially FIG. 4, page 833) and in Haseloff and Gerlach, 1988, Nature, 334:585-591.

According to some embodiments, the ribozyme is engineered so that the cleavage recognition site is located near the 5′ end of the target gene mRNA, e.g., to increase efficiency and minimize the intracellular accumulation of non-functional mRNA transcripts.

The ribozymes of the present invention also include RNA endoribonucleases (hereinafter “Cech-type ribozymes”) such as the ribozyme which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes typically have an eight base pair active site that hybridizes to a target RNA sequence whereafter cleavage of the target RNA takes place.

Ribozymes can be added directly, or can be complexed with cationic lipids, packaged within liposomes, or otherwise delivered to target cells. The RNA or RNA complexes can be locally administered to relevant tissues ex vivo, or in vivo through injection, aerosol inhalation, infusion pump or stent, with or without their incorporation in biopolymers.

Ribozymes can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination can be locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Sullivan, et al., WO 94/02595 which is incorporated herein by reference in its entirety.

Another means of accumulating high concentrations of a ribozyme(s) within cells can involve the incorporation of the ribozyme-encoding sequences into a DNA expression vector. Transcription of the ribozyme sequences can be driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters can also be used, providing that the prokaryotic RNA polymerase enzyme is expressed in the appropriate cells (Elroy-Stein, O. and Moss, B., 1990, Proc. Natl. Acad. Sci. USA, 87, 6743-7; Gao, X. and Huang; L., 1993, Nucleic Acids Res., 21, 2867-72; Lieber, A., et al., 1993, Methods Enzymol., 217, 47-66; Zhou, Y., et al., 1990, Mol. Cell. Biol., 10, 4529-37). Several investigators have demonstrated that ribozymes expressed from such promoters can function in mammalian cells (e.g. (Kashani-Sabet, M., et al., 1992, Antisense Res. Dev., 2, 3-15; Ojwang, J. O., et al., 1992, Proc. Natl. Acad. Sci. USA, 89, 10802-6; Chen, C. J., et al., 1992, Nucleic Acids Res., 20, 4581-9; Yu, M., et al., 1993, Proc. Natl. Acad. Sci. USA, 90, 6340-4; L'Huillier, P. J., et al., 19921, EMBO J., 11, 4411-8; Lisziewicz, J., et al., 1993, Proc. Natl. Acad. Sci. U.S.A., 90, 8000-4)). The above ribozyme transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, Semliki forest virus, sindbis virus vectors).

According to some embodiments, a transcription unit expressing a hairpin ribozyme that cleaves target RNA (e.g., TFF3 mRNA) can be inserted into a plasmid DNA vector or an adenovirus or adeno-associated DNA viral vector. Both viral vectors have been used to transfer genes to the lung and both vectors can lead to transient gene expression (Zabner et al., 1993 Cell 75, 207; Carter, 1992 Curr. Opin. Biotech. 3, 533). The adenovirus vector is delivered as recombinant adenoviral particles. DNA may be delivered alone or complexed with vehicles (as described for RNA above). The DNA, DNA/vehicle complexes, or the recombinant adenovirus particles can be locally administered to the site of treatment, e.g., through the use of an injection catheter, stent or infusion pump or are directly added to cells or tissues ex vivo.

RNAi Molecules

RNA interference (RNAi) is an evolutionarily conserved gene silencing mechanism, originally discovered in studies of the nematode Caenorhabditis elegans (Lee et al, Cell 75:843 (1993); Reinhart et al., Nature 403:901 (2000)). The mechanism is believed to be triggered by introducing dsRNA (double-stranded RNA) into cells expressing the appropriate molecular machinery, which then degrades the corresponding endogenous mRNA. The mechanism involves conversion of dsRNA into short RNAs that direct ribonucleases to homologous mRNA targets (summarized, Ruvkun, Science 2294:797 (2001), which is incorporated herein by reference). This process is related to normal defense against viruses and the mobilization of transposons. Treatment with dsRNA has become a more common method for analyzing gene functions organisms. RNA interference or “RNAi” is a term initially coined by Fire and co-workers to describe the observation that double-stranded RNA (dsRNA) can block gene expression when it is introduced into worms (Fire et al. (1998) Nature 391, 806-811).

The RNAi molecule used to carry out RNAi-mediated modulation of gene expression can comprise one or more strands of polymerized ribonucleotide. It can include any number of modifications such as described above for antisense molecules, including modifications to the phosphate-sugar backbone, the sugar, or the nucleobase to enhance stability or binding affinity. For example, the phosphodiester linkages of natural RNA can be modified to include at least one of a nitrogen or sulfur heteroatom. Modifications in RNA structure can be tailored to allow specific genetic inhibition while avoiding a general panic response in some organisms which is generated by dsRNA. Likewise, bases can be modified to block the activity of adenosine deaminase. RNA may be produced enzymatically or by partial/total organic synthesis. Any modified ribonucleotide can be incorporated into the RNA by in vitro enzymatic or organic synthesis. An RNAi molecule can be a dsRNA molecule.

The size of the RNAi molecule that can be utilized varies according to the size of the subject polynucleotide whose expression is to be suppressed and is sufficiently long to be effective in reducing expression of the subject polynucleotide in a cell. Generally, the RNA is at least about 10 to about 15 nucleotides long. In certain applications, the RNA is less than about 20, 21, 22, 23, 24 or 25 nucleotides in length. In other instances, the RNA is at least about 50, 100, 150 or 200 nucleotides in length. The RNA can be longer still in certain other applications, such as at least about 300, 400, 500 or 600 nucleotides. Typically, the RNA is less than about 3000 nucleotides. The optimal size for any particular subject polynucleotide can be determined by one of ordinary skill in the art without undue experimentation by varying the size of the RNA in a systematic fashion and determining whether the size selected is effective in interfering with expression of the subject polynucleotide.

The RNAi molecule can be introduced in an amount that allows delivery of at least one copy per cell. Higher doses (e.g., at least 5, 10, 100, 500 or 1000 copies per cell) of double-stranded material can yield more effective inhibition; lower doses can also be useful for specific applications. Inhibition is sequence-specific in that nucleotide sequences corresponding to the duplex region of the RNA are targeted for genetic inhibition. Sufficient RNA is introduced into the tissue to cause a detectable change in expression of a target gene (assuming the candidate gene is in fact being expressed in the cell into which the RNA is introduced) using available detection methodologies. Thus, in some instances, sufficient RNA is introduced to achieve at least about 5 to about 10% reduction in candidate gene expression as compared to a cell in which the RNA is not introduced. In other instances, inhibition is at least about 20, 30, 40 or 50%. In still other instances, the inhibition is at least about 60, 70, 80, 90 or 95%. Expression in some instances is essentially completely inhibited to undetectable levels.

The amount of RNA introduced depends upon various factors such as the mode of administration utilized, the size of the RNA, the number of cells into which RNA is administered, and the age and size of an animal if dsRNA is introduced into an animal. An appropriate amount can be determined by those of ordinary skill in the art by initially administering RNA at several different concentrations for example. In certain instances when RNA is introduced into a cell culture, the amount of RNA introduced into the cells can vary from about 0.5 to about 3 μg per 10⁶ cells.

A number of options are available to detect interference of candidate gene expression (i.e., to detect candidate gene silencing). In general, inhibition in expression is detected by detecting a decrease in the level of the protein encoded by the candidate gene, determining the level of mRNA transcribed from the gene and/or detecting a change in phenotype associated with candidate gene expression.

RNA containing a nucleotide sequence substantially identical to a portion of the target gene (e.g., a TFF3 polynucleotide) can be used for RNA inhibition. RNA sequences with insertions, deletions, and single point mutations relative to the target sequence can also be effective for inhibition. Thus, sequence identity, as is well known in the art, can be optimized by sequence comparison and alignment algorithms and calculating the percent difference between the nucleotide sequences by, for example, the Smith-Waterman algorithm as implemented in the BESTFIT software program using default parameters (e.g., University of Wisconsin Genetic Computing Group). In some embodiments, sequence identities can be greater than 90%, or even about 100%, between the inhibitory RNA and the portion of the target gene. Alternatively, the duplex region of the RNA can be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). The length of the RNA nucleotide sequences can be at least about 15, 20, 25, 50, 100, 200, 300 or 400 bases.

As disclosed herein, 100% sequence identity between the RNA and the target gene is not required to practice the present invention. Thus the invention has the advantage of being able to tolerate sequence variations that might be expected due to genetic mutation, strain polymorphism, or evolutionary divergence. However, according to some embodiments, there is 100% sequence identity between the inhibitory RNA and the part of the target gene. Additionally, RNA having greater than about 70%, 80%, 90% or 95% sequence identity may be used in the present invention, and thus sequence variations that might be expected due to genetic mutation, strain polymorphism, or evolutionary divergence can be tolerated.

RNA can be synthesized either in vivo or in vitro. Endogenous RNA polymerase of the cell can mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) can be used to transcribe the RNA strand (or strands). Inhibition can be targeted by specific transcription in an organ, tissue, or cell type; stimulation of an environmental condition (e.g., infection, stress, temperature, chemical inducers); and/or engineering transcription at a developmental stage or age. The RNA strands can be optionally polyadenylated; the RNA strands can be optionally capable of being translated into a polypeptide by a cell's translational apparatus. RNA can further be chemically or enzymatically synthesized by manual or automated reactions. The RNA can be synthesized by a cellular RNA polymerase or a bacteriophage RNA polymerase (e.g., T3, T7, SP6). The use and production of an expression construct are known in the art (See, e.g., WO 97/32016; U.S. Pat. Nos. 5,593,874, 5,698,425, 5,712,135, 5,789,214, and 5,804,693). If synthesized chemically or by in vitro enzymatic synthesis, the RNA can be purified prior to introduction into the cell. For example, RNA can be purified from a mixture by extraction with a solvent or resin, precipitation, electrophoresis, chromatography, or a combination thereof. Alternatively, the RNA can be used with no or a minimum of purification to avoid losses due to sample processing. The RNA can be dried for storage or dissolved in an aqueous solution. The solution can contain buffers or salts to promote annealing, and/or stabilization of the duplex strands.

According to the present invention, RNA having a sequence similar to or substantially identical with a region of sequence of TFF3 polynucleotide can be used to modulate the expression of TFF3 polypeptide in cells such as via an RNA-interference mechanism. The regions of targeted sequence can be selected according to any of the criterion discussed above for antisense modulation of TFF3 expression. In some embodiments, the RNA can include a sequence that is substantially identical with or substantially complementary to any of SEQ ID NOS: 5-19. The RNA can also have a sequence that overlaps with a portion of sequence corresponding to any of SEQ ID NOS: 5-19, or the complement.

RNA can be administered to an organism or patient as described herein for administration of antisense molecules. According to some embodiments, RNA can be directly introduced into the cell (i.e., intracellularly); or introduced extracellularly into a cavity, interstitial space, into the circulation of an organism, introduced orally, or may be introduced by bathing an organism in a solution containing the RNA. Methods for oral introduction include direct mixing of the RNA with food of the organism, as well as engineered approaches in which a species that is used as food is engineered to express the RNA, then fed to the organism to be affected. Physical methods of introducing nucleic acids, for example, injection directly into the cell or extracellular injection into the organism, can also be used. Vascular or extravascular circulation, the blood or lymph system, the phloem, the roots, and the cerebrospinal fluid are sites where the RNA can be introduced. A transgenic organism that expresses RNA from a recombinant construct can be produced by introducing the construct into a zygote, an embryonic stem cell, or another multipotent cell derived from the appropriate organism.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle can accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA can be introduced along with components that perform one or more of the following activities: enhance RNA uptake by the cell, promote annealing of the duplex strands, stabilize the annealed strands, or other-wise increase inhibition of the target gene.

Inhibition of the expression of a target gene can be verified by observing or detecting an absence or observable decrease in the level of protein encoded by a target gene (this may be detected by for example a specific antibody or other techniques known to the skilled person) and/or mRNA product from a target gene (this may be detected by for example hybridization studies) and/or phenotype associated with expression of the gene. In the context of a medical treatment, verification of inhibition of the expression of a target gene can be carried out by observing a change in the disease condition of a subject, such as a reduction in symptoms, remission, a change in the disease state and so on. According to some embodiments, the change in disease state can be slowed tumor growth, reduced tumor volume, reduced motility of cells, reduced invasiveness of cells, and the like. Preferably, the inhibition is specific, i.e. the expression of the target gene is inhibited without manifest effects on the other genes of the cell.

The amount of RNA administered to a mammal for effective gene inhibition can vary according to a wide variety of factors, including the route of administration, the age, size and condition of the mammal, the gene which is to be inhibited, the disease or disorder to be treated and so on. According to some embodiments, the dsRNA can be administered to provide 0.1 to 400 pg, preferably 1 to 40 pg and most preferably 1 to 20 pg in each cell.

Methods for using RNAi, either exogenous addition or transcription in vivo, are known in the art (see Schubiger and Edgar, Methods in Cell Biology (1994) 44:697-713, and PCT application WO 99/32619, respectively, each of which is incorporated herein by reference), For example, in C. elegans, RNAi has been shown to knock out a tumor suppressor gene in vulval precursor cells (Lu and Horvitz, Cell (1998) 95:981-991, which incorporated herein by reference in its entirety).

Methods for reducing or suppressing expression of certain gene products using RNAi is further provided in U.S. Pat. No. 6,506,559 and U.S. Pat. Application Pub. No. 20030027783 (related to inhibiting gene expression in mammals), each of which is incorporated by reference in its entirety.

Antibodies

The neutralizing agent can also be an immunoglobin such as an antibody or fragment thereof that binds to a TFF3 protein. Immunoglobulins include both antibodies and other antibody-like molecules that lack antigen specificity. Polypeptides of the latter kind are, for example, produced at low levels by the lymph system and at increased levels by myelomas.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, intact antibodies can be assigned to different “classes.”

Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy chain variable domains (Chothia et al. J. Mol. Biol. 186:651 (1985; Novotny and Haber, Proc. Natl. Acad. Sci. U.S.A. 30 82:4592 (1985); Chothia et al., Nature 342:877-883 (1989)).

The term “antibody” is used in the broadest sense and covers fully assembled polyclonal and monoclonal antibodies, as well as antibody fragments that can bind antigen (e.g., Fab′, F′(ab)₂, Fv, single chain antibodies, diabodies).

The term “monoclonal antibody” as used herein refers to an antibody obtained from a population of substantially homogeneous antibodies, i.e., the individual antibodies comprising the population are identical except for possible naturally-occurring mutations that may be present in minor amounts.

A description follows as to exemplary techniques for the production of the antibody neutralizing agents used in accordance with the present invention.

Antibodies are said to be “specifically binding” if: 1) they exhibit a threshold level of binding activity, and/or 2) they do not significantly cross-react with known related polypeptide molecules. The binding affinity of an antibody can be readily determined by one of ordinary skill in the art, for example, by Scatchard analysis (Scatchard, Ann. NY Acad. Sci. 51: 660-672, 1949). In some embodiments, the antibodies of the present invention bind to TFF3 at least 10³, more preferably at least 10⁴, more preferably at least 10⁵, and even more preferably at least 10⁶ fold higher than to other proteins related to TFF3.

In some embodiments, the antibodies of the present invention do not specifically bind to (or recognize) known related polypeptide molecules, for example, if they bind TFF3 polypeptide but not known related polypeptides using a standard Western blot analysis (Ausubel et al.). In some embodiments antibodies may be screened against known related polypeptides to isolate an antibody population that specifically binds to TFF3 polypeptides. For example, antibodies specific to TFF3 polypeptides will flow through a column comprising TFF3 family polypeptides (with the exception of TFF3) adhered to insoluble matrix under appropriate buffer conditions. Such screening allows isolation of polyclonal and monoclonal antibodies non-crossreactive to closely related polypeptides (Antibodies: A Laboratory Manual, Harlow and Lane (eds.), Cold Spring Harbor Laboratory Press, 1988; Current Protocols in Immunology, Cooligan et al. (eds.), National Institutes of Health, John Wiley and Sons, Inc., 1995). Screening and isolation of specific antibodies is well known in the art (see, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984). Representative examples of such assays include: concurrent immunoelectrophoresis, radioimmunoassay (RIA), radioimmunoprecipitation, enzyme-linked immunosorbent assay (ELISA), dot blot or Western blot assay, inhibition or competition assay, and sandwich assay.

In some embodiments, the antibodies of the present invention have at least about 1000 fold, and at least about 10,000 fold greater affinity for TFF3 than for known related family members. In some embodiments, the binding affinity of an antibody of the present invention is less than about 1×10⁵ Kₐ, less than about 1×10⁴ Kₐ, and preferably less than 1×10³ Kₐ, for a related polypeptide other than TFF3.

Polyclonal Antibodies

Polyclonal antibodies can be raised in animals by multiple subcutaneous (sc), intraperitoneal (ip) or intramuscular (im) injections of the relevant antigen and an adjuvant. It can be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups. Animals are immunized against the antigen, immunogenic conjugates, or derivatives by combining, e.g., 100 pg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal Antibodies

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. For example, the monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animals, such as a rabbit or hamster, is immunized as herein above described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell [Goding, Monoclonal Antibodies: Principles and Practice, pp. 59-103 (Academic Press, 1986)].

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Examplery myeloma cells are those that fuse efficiently, support stable high-level production of antibody by the selected antibody-producing cells, and are sensitive to a medium such as HAT medium, including myeloma cell lines such as murine myeloma lines, including those derived from MOPC-21 and MPC-11 mouse tumors available from the Salk Institute Cell Distribution Center, San Diego, Calif. USA, and SP-2, NZ0, or X63-Ag8-653 cells available from the American Type Culture Collection, Rockville, Md. USA. Human myeloma and mouse human heteromyeloma cell lines also have been described for the production of human monoclonal antibodies [Kozbor, J. Immunol., 133:3001 (1984); Brodeur et al., Monoclonal Antibody Production Techniques and Applications, pp. 51-63 (Marcel Dekker, Inc., New York, 1987)].

Culture medium in which hybridoma cells are growing is assayed for the production of monoclonal antibodies having the requisite specificity, e.g. by an in vitro binding assay such as enzyme-linked immunoabsorbent assay (ELISA) or radioimmunoassay (RIA). The location of the cells that express the antibody may be detected by FACS. Thereafter, hybridoma clones may be subcloned by limiting dilution procedures and grown by standard methods (Goding, Monoclonal Antibodies: Principles and Practice, Academic Press (1986) pp. 59-103). Suitable culture media for this purpose include, for example, DMEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Phickthun, Immunol. Revs., 130:151-188 (1992).

In further embodiments, antibodies or antibody fragments are isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991), describe the isolation of murine and human antibodies, respectively, using phage libraries. Subsequent publications describe the production of high affinity (nM range) human antibodies by chain shuffling (Marks et al., Bio/Technology, 10:779-783 (1992) which is incorporated herein by reference in its entirety), as well as combinatorial infection and in vivo recombination as a strategy for constructing very large phage libraries (Waterhouse et al., Nuc. Acids. Res., 21:2265-2266 (1993) which is incorporated herein by reference in its entirety). Thus, these techniques are viable alternatives to traditional monoclonal antibody hybridoma techniques for isolation of monoclonal antibodies.

DNA also may be modified, for example, by substituting the coding sequence for human heavy- and light-chain constant domains in place of the homologous murine sequences (U.S. Pat. No. 4,816,567; Morrison, et al, Proc. Natl. Acad. Sci. USA, 81:6851 (1984) which is incorporated herein by reference in its entirety), or by covalently joining to the immunoglobulin coding sequence all or part of the coding sequence for a non-immunoglobulin polypeptide. Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen combining site having specificity for a different antigen.

Additionally, recombinant antibodies against TFF3 can be produced in transgenic animals, e.g., as described in various patents. For example, recombinant antibodies can be expressed in transgenic animals, e.g., rodents as disclosed in any of U.S. Pat. Nos. 5,877,397, 5,874,299, 5,814,318, 5,789,650, 5,770,429, 5,661,016, 5,633,425, 5,625,126, 5,569,825, 5,545,806, 6,162,963, 6,150,584, 6,130,364, 6,114,598, 6,091,001, 5,939,598. Alternatively, recombinant antibodies can be expressed in the milk of transgenic animals as discussed in U.S. Pat. No. 5,849,992 or 5,827,690.

Humanized Antibodies

Methods for humanizing non-human antibodies have been described in the art. Preferably, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al, Nature, 332:323-327 (1988); Verhoeyen et al, Science, 239:1534-1536 (1988)), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies affects antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al, J. Immunol, 151:2296 (1993); Chothia et al., J. Mol. Biol, 196:901 (1987)). Another method uses a particular framework region derived from the consensus sequence of all human antibodies of a particular subgroup of light or heavy chains. The same framework may be used for several different humanized antibodies (Carter et al., Proc. Nad. Acad. Sci. USA, 89:4285 (1992); Presta et al., J. Immunol, 151:2623 (1993)).

Antibodies can be humanized with retention of high affinity for the antigen and other favorable biological properties. To achieve this goal, according to some embodiments, humanized antibodies are prepared by a process of analysis of the parental sequences and various conceptual humanized products using three dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Human Antibodies

As an alternative to humanization, human antibodies can be generated. As discussed above, the production of antibodies, particularly human antibodies in transgenic animals is known. For example, transgenic animals (e.g., mice) can be produced that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Mad. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807. Alternatively, phage display technology (McCafferty et al., Nature 348:552-553 (1990) which is incorporated herein by reference in its entirety) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g. Johnson, Kevin S. and Chiswell, David J., Current Opinion in Structural Biology 3:564-571(1993). Several sources of V-gene segments can be used for phage display. Clackson et al., Nature, 352: 624-628 (1991), isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905. Human antibodies may also be generated by in vitro activated B cells (see U.S. Pat. Nos. 5,567,610 and 5,229,275).

Antibody Fragments

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 (1985)). However, these fragments can also be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments [Carter et al., Bio/Technology 10:163-167 (1992) which is incorporated herein by reference in its entirety]. According to another approach, F(ab′)₂ fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment may also be a “linear antibody”, e.g., as described in U.S. Pat. No. 5,641,870, for example. Such linear antibody fragments may be monospecific or bispecific. Moreover, the nucleic acids encoding the antibody fragments identified in phage display libraries can be cloned, sequenced, and engineered to be part of nucleic acid that encodes and can express a full sized antibody of any isotype.

Bispecific Antibodies

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983) which is incorporated herein by reference in its entirety). Because of the random assortment of immunoglobulin heavy and light chains, these hybridomas (quadromas) produce a potential mixture of 10 different antibody molecules, of which only one has the correct bispecific structure. Purification of the correct molecule, which is can be performed by affinity chromatography steps, is rather cumbersome, and the product yields are low. Similar procedures are disclosed in WO 93/08829, and in Traunecker et al., EMBO J, 10:3655-3659 (1991).

According to a different approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. In some embodiments, the first heavy-chain constant region (CH1) containing the site necessary for light chain binding is present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some embodiments bispecific antibodies contain a hybrid immunoglobulin heavy chain with a first binding specificity in one arm, and a hybrid immunoglobulin heavy chain light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology, 121:210(1986).

According to another approach described in U.S. Pat. No. 5,731,168, the interface between a pair of antibody molecules can be engineered to maximize the percentage of heterodimers which are recovered from recombinant cell culture. In some embodiments the interface comprises at least a part of the CH3 domain of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360 and WO 92/200373). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229:81 (1985), describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equivalent amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med., 175: 217-225 (1992), describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993), has provided an alternative mechanism for making bispecific antibody fragments. The fragments comprise a heavy-chain variable domain (VH) connected to a light-chain variable domain (VL) by a linker which is too short to allow pairing between the two domains on the same chain.

Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. See Gruber et al., J. Immunol., 152:5368 (1994). Antibodies with more than two valencies are contemplated. For example, trispecific antibodies can be prepared. Tutt et al. J. Immunol. 147: 60 (1991).

Conjugates and Other Modifications of the Neutralizing Agent

The neutralizing agents used in the methods or included in the articles of manufacture herein can be optionally conjugated to a cytotoxic or therapeutic agent. Examples include chemotherapeutic agents. Such chemotherapeutics can have an established efficacy in treatment of a particular cancer.

Conjugates of a neutralizing agent and one or more small molecule toxins, such as a calicheamicin, a maytansine (U.S. Pat. No. 5,208,020), a trichothene, and CC1065 are also contemplated herein. According to some embodiments, the neutralizing agent is conjugated to one or more maytansine molecules (e.g. about 1 to about 10 maytansinemolecules per neutralizing agent molecule). Maytansine may, for example, be converted to May-SS-Me which may be reduced to May-SH3 and reacted with modified neutralizing agent (Chari et al. Cancer Research 52: 127-131 (1992)) to generate a maytansinoid-neutralizing agent conjugate.

Alternatively, the neutralizing agent is conjugated to one or more calicheamicin molecules. The calicheamicin family of antibiotics are capable of producing double-stranded DNA breaks at sub-picomolar concentrations. Structural analogues of calicheamicin are also known. (Hinman et al. Cancer Research 53: 3336-3342 (1993) and Lode et al. Cancer Research 58: 2925-2928 (1998)).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232,

The present invention further contemplates a neutralizing agent conjugated with a compound having nucleolytic activity (e.g. a ribonuclease or a DNA endonuclease such as a deoxyribonuclease; DNase). A variety of radioactive isotopes are available for the production of radioconjugated neutralizing agents. Examples include Y⁹⁰, At²¹¹, Re¹⁸⁶, Re¹⁸⁸, Sm¹⁵³, Bi²¹², p³² and radioactive isotopes of Lu. Conjugates of the neutralizing agent and cytotoxic agent may be made using a variety of bifunctional protein coupling agents such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aidehydes (such as glutareldehyde), bis azido compounds (such as bis (p-azidobenzoyl) hexanediamine), bis-diazonium derivatives (such as bis-(pdiazoniumbenzoyl)-ethylenediamine), diisocyanates (such as tolyene 2,6-diisocyanate), and bis-active fluorine compounds (such as 1,5-difluoro-2, 4-dinitrobenzene). For example, a ricin immunotoxin can be prepared as described in Vitetta et al. Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the neutralizing agent. See, for example, WO94/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52: 127-131 (1992)) may be used. Alternatively, a fusion protein comprising the neutralizing agent and cytotoxic agent may be made, e.g. by recombinant techniques or peptide synthesis.

In some embodiments, the neutralizing agent can be conjugated to a “receptor” (such streptavidin) for utilization in tumor pretargeting wherein the neutralizing agent-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). The neutralizing agents of the present invention can also be conjugated with a prodrug-activating enzyme which converts a prodrug (e.g. a peptidyl chemotherapeutic agent, see WO81/01145) to an active anti-cancer drug. See, for example, WO 88/07378 and U.S. Pat. No. 4,975,278.

The enzyme component of such conjugates includes any enzyme capable of acting on a prodrug in such a way so as to covert it into its more active, cytotoxic form. Enzymes that are useful in the method of this invention include, but are not limited to, alkaline phosphatase useful for converting phosphate-containing prodrugs into free drugs; arylsulfatase useful for converting sulfate containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate cleaving enzymes such as β-galactosidase and neuraminidase useful for converting glycosylated prodrugs into free drugs; (3-lactamase useful for converting drugs derivatized with (3-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). Neutralizing agent-abzyme conjugates can be prepared as described herein for delivery of the abzyme to a tumor cell population.

Enzymes can be covalently bound to the TFF3 neutralizing agent by techniques well known in the art such as the use of the heterobifunctional crosslinking reagents discussed above. Alternatively, fusion proteins comprising at least the antigen binding region of an neutralizing agent of the invention linked to at least a functionally active portion of an enzyme of the invention can be constructed using recombinant DNA techniques well known in the art [see, e.g., Neuberger et al., Nature, 312: 604-608 (1984)].

Other modifications of the neutralizing agent are contemplated herein. For example, the neutralizing agent can be linked to one of a variety of nonproteinaceous polymers, e.g., polyethylene glycol, polypropylene glycol, polyoxyalkylenes, or copolymers of polyethylene glycol and polypropylene glycol. The neutralizing agents disclosed herein may also be formulated as liposomes. Liposomes containing the neutralizing agent are prepared by methods known in the art, such as described in Epstein et al., Proc. Mad. Acad. Sci. USA, 82:3688 (1985); Hwang et al., Proc. Natl. Acad. Sci. USA, 77:4030 (1980); U.S. Pat. Nos. 4,485,045 and 4,544,545; and WO97/38731 Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to the liposomes as described in Martin et al., J. Biol. Chem. 257: 286-288 (1982), via a disulfide interchange reaction. A chemotherapeutic agent is optionally contained within the liposome. See Gabizon, et al. J. National Cancer Inst. 81(19)1484 (1989). Amino acid sequence modification(s) of protein or peptide neutralizing agents described herein are contemplated. For example, it may be desirable to improve the binding affinity and/or other biological properties of the neutralizing agent.

Amino acid sequence variants of the neutralizing agent are prepared by introducing appropriate nucleotide changes into the neutralizing agent nucleic acid, or by peptide synthesis. Such modifications include, without limitation, deletions from, and/or insertions into and/or substitutions of, residues within the amino acid sequences of the neutralizing agent. Any combination of deletion, insertion, and substitution is made to arrive at the formal construct, provided that the final construct possesses the desired characteristics. The amino acid changes also may alter post-translational processes of the neutralizing agent, such as changing the number or position of glycosylation sites.

A useful method for the identification of certain residues or regions of the neutralizing agent that are locations for mutagenesis is called “alanine scanning mutagenesis” as described by Cunningham and Wells, Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed neutralizing agent variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an neutralizing agent with an N-terminal methionyl residue or the neutralizing agent fused to a cytotoxic polypeptide. Other insertional variants of the neutralizing agent molecule include the fusion to the N- or C-terminus of the neutralizing agent of an enzyme, or a polypeptide which increases the serum half-life of the neutralizing agent.

Another type of variant is an amino acid substitution variant. These variants have at least one amino acid residue in the neutralizing agent molecule replaced by different residue. The sites of greatest interest for substitutional mutagenesis of antibody neutralizing agents include the hypervariable regions, but FR alterations are also contemplated.

Substantial modifications in the biological properties of the neutralizing agent are accomplished by selecting substitutions that differ significantly in their effect on maintaining (i) the structure of the polypeptide backbone in the area of the substitution, for example, as a sheet or helical conformation, (ii) the charge or hydrophobicity of the molecule at the target site, or (iii) the bulk of the side chain. Naturally occurring residues are divided into groups based on common side-chain properties:
hydrophobic: norleucine, met, ala, val, leu, ile;
neutral hydrophilic: cys, ser, thr;
acidic: asp, glu;
basic: asn, gin, his, lys, arg;
residues that influence chain orientation: gly, pro; and
aromatic: trp, tyr, phe.

Non-conservative substitutions will entail exchanging a member of one of these classes for another class. Conservative substitutions involve exchanging of amino acids within the same class.

Any cysteine residue not involved in maintaining the proper conformation of the neutralizing agent also may be substituted, generally with serine, to improve the oxidative stability of the molecule and prevent aberrant cross-linking. Conversely, cysteine bond(s) may be added to the neutralizing agent to improve its stability (particularly where the neutralizing agent is an antibody fragment such as an Fv fragment).

In some embodiments, a type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in addition, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the neutralizing agent described herein alters the original glycosylation pattern of the neutralizing agent. By “altering” is meant deleting one or more carbohydrate moieties found in the neutralizing agent, and/or adding one or more glycosylation sites that are not present in the neutralizing agent.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. Addition of glycosylation sites to the neutralizing agent is accomplished by altering the amino acid sequence such that it contains one or more of the above-described tripeptide sequences (for N-linked glycosylation sites). The alteration may also be made by the addition of, or substitution by, one or more serine or threonine residues to the sequence of the original neutralizing agent (for O-linked glycosylation sites).

Nucleic acid molecules encoding amino acid sequence variants of the neutralizing agent are prepared by a variety of methods known in the art. These methods include, but are not limited to, isolation from a natural source (in the case of naturally occurring amino acid sequence variants) or preparation by oligonucleotide-mediated (or site directed) mutagenesis, PCR mutagenesis, and cassette mutagenesis of an earlier prepared variant or a non-variant version of the neutralizing agent.

In some embodiments, it may be desirable to modify the neutralizing agent of the invention with respect to effector function, e.g. so as to enhance antigen-dependent cell-mediated cyotoxicity (ADCC) and/or complement dependent cytotoxicity (CDC) of the neutralizing agent. This may be achieved by introducing one or more amino acid substitutions in an Fc region of an antibody neutralizing agent. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med 176:1191-1195 (1992) and Shopes, B. J. Immunol. 148:2918-2922 (1992). Homodimeric antibodies with enhanced anti-tumor activity may also be prepared using heterobifunetional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989).

To increase the serum half life of the neutralizing agent, a salvage receptor binding epitope can be incorporated into the neutralizing agent (especially an antibody fragment) as described in, for example, U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

The present invention also provides screens for identifying anti-TFF3 antibodies having desirable therapeutic or diagnostic properties. Screens include assays that identify anti-TFF3 antibodies which affect proliferation and/or adhesion of tumor cells, ADCC or CDC activity, anti-apoptotic assays, cell cycle checkpoint assays, and in vivo assays in transgenic non-human animals, e.g., mice and other rodents. Also the present invention contemplates screens to identify antibodies that bind to desired portions of the protein as described supra, and which possess desired properties, e.g., block calcium binding, block dimer formation, block strand formation and/or interfere with TFF3 domain alignment. Such antibodies can be identified for populations of antibodies provided against TFF3 protein or fragments thereof.

Other Forms of Neutralizing Agents

Neutralizing agents can also be in the form of a prodrug. The term “prodrug” refers to a therapeutic agent that is prepared in an inactive form that is converted to an active form (i.e., drug) within the body or cells thereof by the action of endogenous enzymes or other chemicals and/or conditions. In particular, prodrug versions of nucleic acid-containing neutralizing agents (such as antisense oligonucleotides, ribozymes, and RNAi molecules, and the like) can be prepared as SATE [(S-acetyl-2-thioethyl) phosphate] derivatives according to the methods disclosed in WO 93/24510 or in WO 94/26764 and U.S. Pat. No. 5,770,713.

Neutralizing agents can also be in the form of pharmaceutically acceptable salts. The term “pharmaceutically acceptable salts” refers to physiologically and pharmaceutically acceptable salts of the compounds of the invention: i.e., salts that retain the desired biological activity of the parent compound and do not impart undesired toxicological effects thereto. For nucleic acid-containing neutralizing agents (e.g., antisense oligonucleotides, ribozymes, RNAi molecules, and the like), some examples of pharmaceutically acceptable salts include, but are not limited to, (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

Polynucleotide Constructs

Polynucleotide molecules encoding TFF3, a TFF3 fragment, or a TFF3 neutralizing agent such as an antibody can be inserted into a polynucleotide construct, such as a DNA or RNA construct. Polynucleotide molecules of the invention can be used, for example, in an expression construct to express all or a portion of a protein, variant, fusion protein, or single-chain antibody in a host cell. An expression construct comprises a promoter that is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment that encodes all or a portion of the desired protein. The polynucleotide segment is located downstream from the promoter. Transcription of the polynucleotide segment initiates at the promoter. The expression construct can be linear or circular and can contain sequences, if desired, for autonomous replication.

Host Cells

An expression construct encoding TFF3 or a TFF3 neutralizing agent can be introduced into a host cell. The host cell comprising the expression construct can be any suitable prokaryotic or eukaryotic cell. Expression systems in bacteria include those described in Chang et al., Nature 275:615 (1978); Goeddel et al., Nature 281: 544 (1979); Goeddel et al., Nucleic Acids Res. 8:4057 (1980); EP 36,776; U.S. Pat. No. 4,551,433; deBoer et al., Proc. Natl. Acad. Sci. USA 80:21-25(1983); and Siebenlist et al., Cell 20:269(1980).

Expression systems in yeast include those described in Hinnnen et al., Proc. Natl. Acad. Sci. USA 75:1929 (1978); Ito et al., J Bacteriol 153:163 (1983); Kurtz et al., Mol. Cell. Biol. 6:142 (1986); Kunze et al., J Basic Microbiol. 25:141 (1985); Gleeson et al., J. Gen. Microbiol. 132: 3459 (1986), Roggenkamp et al., Mol. Gen. Genet. 202: 302 (1986)); Das et al., J. Bacteriol. 158: 1165 (1984); De Louvencourt et al., J. Bacteriol. 154:737 (1983), Van den Berg et al., Bio/Technology 8: 135 (1990); Kunze et al., J. Basic Microbiol. 25: 141 (1985); Cregg et al., Mol. Cell. Biol. 5: 3376 (1985); U.S. Pat. No. 4,837,148; U.S. Pat. No. 4,929,555; Beach and Nurse, Nature 300: 706 (1981); Davidow et al., Curr. Genet. 10: 380 (1985); Gaillardin et al., Curr. Genet. 10: 49 (1985); Ballance et al., Biochem. Biophys. Res. Commun. 112: 284-289 (1983); Tilburn et al., Gene 26: 205-22 (1983); Yelton et al., Proc. Natl. Acad, Sci. USA 81: 1470-1474 (1984); Kelly and Hynes, EMBO J. 4: 475479 (1985); EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. 69: 765-776 (1988); Miller et al., Ann. Rev. Microbiol. 42: 177 (1988); Carbonell et al., Gene 73: 409 (1988); Maeda et al., Nature 315: 592-594 (1985); Lebacq-Verheyden et al., Mol. Cell Biol. 8: 3129 (1988); Smith et al., Proc. Natl. Acad. Sci. USA 82: 8404 (1985); Miyajima et al., Gene 58: 273 (1987); and Martin et al., DNA 7:99 (1988). Numerous baculoviral strains and variants and corresponding permissive insect host cells from hosts are described in Luckow et al., Bio/Technology (1988) 6: 47-55, Miller et al., in GENETIC ENGINEERING (Setlow, J. K. et al. eds.), Vol. 8, pp. 277-279 (Plenum Publishing, 1986); and Maeda et al., Nature, 315: 592-594 (1985).

Mammalian expression can be accomplished as described in Dijkema et al., EMBO J. 4: 761(1985); Gormanetal., Proc. Natl. Acad. Sci. USA 79: 6777 (1982b); Boshart et al., Cell 41: 521 (1985); and U.S. Pat. No. 4,399,216. Other features of mammalian expression can be facilitated as described in Ham and Wallace, Meth Enz. 58: 44 (1979); Barnes and Sato, Anal. Biochem. 102: 255 (1980); U.S. Pat. No. 4,767,704; U.S. Pat. No. 4,657,866; U.S. Pat. No. 4,927,762; U.S. Pat. No. 4,560,655; WO 90/103430, WO 87/00195, and U.S. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Pharmaceutical Formulations

Therapeutic formulations of the TFF3 neutralizing agents in accordance with the present invention can be prepared for storage by mixing a neutralizing agent having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt forming counter-ions such as sodium; metal complexes (e.g. Zn-protein complexes); and/or non-ionic surfactants such as TWEEN™, PLURONICS™ or polyethylene glycol (PEG). Acceptable carriers, excipients, or stabilizers further do not interfere with the activity of the neutralizing agent.

Formulations can also contain more than one active compound. In some embodiments, active compounds have complementary activities that do not adversely affect each other. For example, it may be desirable to further provide a cytotoxic agent, chemotherapeutic agent or cytokine. The effective amount of such other agents depends on the amount of neutralizing agent present in the formulation, the type of cancer treatment, and other factors discussed above. These are generally used in the same dosages and with administration routes as used herein before or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations can also be prepared. Suitable examples of sustained-release preparations include semipermeable matrices of solid hydrophobic polymers containing the neutralizing agent, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid glycolic acid copolymer and leuprolide acetate), and poly-D-(−)-3-hydroxybutyric acid.

In some embodiments, administration can be through the use of sterile injectable pharmaceutical compositions. As used herein, the phrase “injectable pharmaceutical composition”, or variants thereof, refers to pharmaceutical compositions which satisfy the USP requirements for “injectables”, i.e., sterile, pyrogen- and particulate free, and possessing specific pH and isotonicity values. Sterilizing solution formulations can be accomplished by filtration through sterile filtration membranes.

Treatment with the Neutralizing Agent

The treatment or prevention of cancer can be effected by administering a therapeutically effective amount of a TFF3 neutralizing agent to a mammal (e.g., human) suffering from or predisposed to cancer. Likewise, methods for reducing tumor volume, slowing tumor growth, and/or preventing tumor growth can be achieved by similar means.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or relieving the disease symptom, i.e., causing regression of the disease or symptom, such as colon or another digestive cancer, e.g., stomach or liver, or breast, ovarian, or prostate cancer.

The terms “individual,” “subject,” “host,” and “patient,” used interchangeably and refer to any mammalian subject for whom diagnosis, treatment, or therapy is desired, particularly humans. Other subjects may include cattle, dogs, cats, guinea pigs, rabbits, rats, mice, horses, and the like.

A composition comprising a TFF3 neutralizing agent, e.g. an antibody, peptide, antisense molecule, RNAi molecule, ribozyme, or small molecule can be formulated, dosed, and administered in a fashion consistent with good medical practice. For example, the TFF3 neutralizing agent is a human, chimeric or humanized anti-TFF3 antibody scFv, antibody fragment, peptide, or small molecule that inhibits activity of TFF3; or an antisense oligonucleotide, RNAi molecule, or ribozyme that inhibits TFF3 expression. Factors for consideration in this context include the particular cancer being treated, the particular mammal being treated, the clinical condition of the individual patient, the cause of the disease or disorder, the site of delivery of the agent, the method of administration, the scheduling of administration, and other factors known to medical practitioners. The therapeutically effective amount of the neutralizing agent to be administered can be governed by such considerations.

The therapeutically effective amount of a neutralizing agent is an amount sufficient to ameliorate or lessen the symptoms associated with a disease or disorder in a patient treated with the neutralizing agent. In some embodiments, a therapeutically effective amount is an amount of neutralizing agent that can lessen the symptoms of cancer, such as by preventing tumor growth, slowing tumor growth, reducing tumor volume, reducing the invasiveness of cancer cell, inhibiting the migration, adhesion, and/or proliferation of cancer cells, and the like. Methods of determining a therapeutically effective amount and evaluating therapeutic effects of a neutralizing agent are known in the art and further described herein.

A therapeutically effective amount administered parenterally per dose can be, for example, in the range of about 0.1 to 30 mg/kg of patient body weight per day, with the typical initial range of neutralizing agent used being in the range of about 2 to 10 mg/kg. As noted above, however, these suggested amounts of neutralizing agent can be subject to a great deal of therapeutic discretion. A factor in selecting an appropriate dose and scheduling is the result obtained, as indicated above. For example, relatively higher doses may be needed initially for the treatment of ongoing and acute diseases. To obtain the most efficacious results, depending on the disease or disorder, the neutralizing agent is administered as close to the first sign, diagnosis, appearance, or occurrence of the disease or disorder as possible or during remissions of the disease or disorder.

The neutralizing agent can be administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration.

In addition, the neutralizing agent can be suitably administered by pulse infusion, e.g., with declining doses of the neutralizing agent. Preferably the dosing is given by injections, most preferably intravenous or subcutaneous injections, depending in part on whether the administration is brief or chronic.

One can administer other compounds, such as cytotoxic agents, chemotherapeutic agents, immunosuppressive agents and/or cytokines with the neutralizing agents herein. The combined administration includes coadministration, using separate formulations or a single pharmaceutical formulation, and consecutive administration in either order, wherein preferably there is a time period while both (or all) active agents simultaneously exert their biological activities.

There are numerous approaches in the art for inserting a nucleic acid, such as an antisense molecule, RNAi molecule, or ribozyme (optionally contained in a vector) into the patient's cells; in vivo and ex vivo. For in vivo delivery the nucleic acid can be injected directly into the patient, usually at the site where the neutralizing agent is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus. Nucleic acids can also be administered by hydrodynamic delivery (increased pressure intravascular injection).

An example of in vivo nucleic acid transfer technique includes transfection with viral vectors (such as adenovirus, Herpes simplex I virus, or adeno-associated virus) and lipid-based systems (useful lipids for lipid mediated transfer of the gene are DOTMA, DOPE and DC-Choi, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. USA 87:3410-3414 (1990).

In accordance with the present invention, treatment can also inlude combination therapy. As used herein “combination therapy” means that the patient in need of a drug is treated or given another drug for the disease in conjunction with a neutralizing agent. This combination therapy can be sequential therapy where the patient is treated first with one or more drugs and then the other, or two or more drugs are given simultaneously. Some example drugs that can be used in combination therapy include chemotherapeutic agents (cisplatin, doxirubicin, danurubicin, tamoxiphen, taxol, methotrexate, etc.), aromatase inhibitors, administration of angiogenesis inhibitors, specific and nonspecific immunomodulating agents, biological response modifiers (BRMs), colony-stimulating factors (CSFs), interferons, interleukins, autologous tumor cell vaccines, hormones, and the like. Preferably, the drugs or other agents administered in combination do not interfere with the therapeutic activity of the neutralizing agent.

In some embodiments, administration of a neutralizing agent can be combined with traditional cancer treatments. Preferably, the traditional cancer treatment does not interfere with or reduce the effectiveness of the neutralizing agent. Some example traditional cancer treatments include surgery (including, e.g., cryosurgery, segmental resection surgery, radical prostatectomy, lumpectomy, mastectomy, etc.), chemotherapy, radiation therapy (e.g., internal radiation therapy, external beam radiation therapy), brachytherapy (e.g., delivery of radiation directly to the original tumor site and decrease radiation time using a single catheter to perform a breast cancer therapy), hormone ablation therapy (reduction of hormone levels), and the like.

The present invention further provides methods of reducing tumor volume, slowing tumor growth, and/or preventing tumor growth by contacting the tumor with a TFF3 neutralizing agent. The contacting can be carried out, for example, in vivo where the tumor is exposed to a TFF3 neutralizing agent by any suitable means. For example, the tumor can be directly injected or coated with a composition containing a TFF3 neutralizing agent, or a subject or patient having the tumor can be administered a TFF3 neutralizing agent. Tumor volume and growth rates can be readily measured by methods known in the art.

Additionally, the physiological effects of TFF3 (such as overexpressed TFF3) in a tissue or cell can be modulated by administration of a TFF3 neutralizing agent to a patient or by contacting cells with a TFF3 neutralizing agent. Several physiological effects due to expression of TFF3 are discussed hereinabove, and include, for example, increased cell motility (e.g., migratory ability) and resistance to apoptosis.

Cell motitility, migration, and potential invasiveness can be assessed by an assay involving wounding a confluent plate of cells and measuring migration of cells into the wound (e.g., either by time-lapse video microscopy or by time to fill in the wound). Two other assays make use of transwell filters, in which cells are plated on the top of a porous transwell insert and the number of cells migrating to the bottom well or onto the underside of a fibronection coated filter are counted. These latter two assays are a modification of the “boyden-chamber” assay. The transwell assays can be further modified to measure chemotaxis (by putting a chemoattractant in the bottom well), chemokinesis (by putting a motility inducing chemical in both the top and bottom wells) and invasion (by coating the well with reconstituted extracellular matrix, such as matrigel).

Apoptosis or resistance thereto can be measured by any general cytoxicity assay. Many methods are known in the art such as cell morphology, appearance of the characteristic 180 bp DNA ladder banding pattern on agarose gels, the TUNEL (TdT-mediated dUTP Nick-End Labeling) assay, flow cytometry, DNA fragment ELISA, and changes in the biophysical properties of the cell membrane. The caspase family of cysteine proteases have also been identified as common mediators of the cell suicide pathway and assays for caspase activity have been added to the arsenal of methods used to detect apoptosis. A lactate dehydrogenase (LDH) leakage assay, can also be used to detect or measure apoptosis. Kits for carrying out many of these methods are available commericially.

Cells, such as those differentially expressing TFF3, can be contacted with a TFF3 neutralizing agent in vitro, in vivo, or ex vivo. Contacting can be achieved by any of the formulation/administration methods described herein or, for example, by exposing cells to a medium containing a TFF3 neutralizing agent. For example, cells can be washed, incubated, or suspended in a solution or agar medium for an amount of time sufficient for the TFF3 neutralizing agent to at least partially penetrate the cell membrane and/or come in contact with a TFF3 polypeptide or TFF3 polynucleotide in the cell. Formulation, Dosage, Pharmaceutical Compositions.

The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, caplets, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In some embodiments, the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

A “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration that do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances that increase the viscosity of the suspension including, for example, sodium carboxymethylcellulose, sorbitol and/or dextran. The suspension may also contain stabilizers.

Some embodiments of the invention provide pharmaceutical compositions comprising: (a) one or more antisense compounds and (b) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). See, generally, The Merck Manual of Diagnosis and Therapy, 15th Ed., Berkow et al., eds., 1987, Rahway, N.J., pages 1206-1228). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively). Other non-antisense chemotherapeutic agents are also within the scope of this invention. Two or more combined compounds may be used together or sequentially.

Diagnosis, Prognosis, Assessment of Therapy (Therametrics), and Management of Cancer

The TFF3 polynucleotides described herein, as well as their gene products, are of further interest as genetic or biochemical markers (e.g., in blood or tissues) that can detect the earliest changes along the carcinogenesis pathway and/or to monitor the efficacy of various therapies and preventive interventions. For example, the level of expression of TFF3 can be indicative of a poorer prognosis, and therefore warrant more aggressive chemotherapy or radiotherapy for a patient or vice versa. The correlation of novel surrogate tumor specific features with response to treatment and outcome in patients can define prognostic indicators that allow the design of tailored therapy based on the molecular profile of the tumor. These therapies include antibody targeting, neutralizing agents (e.g., small molecules), and gene therapy. Determining expression of TFF3 and comparing a patient's profile with known expression in normal tissue and variants of the disease may allow a determination of the best possible treatment for a patient, both in terms of specificity of treatment and in terms of comfort level of the patient. Surrogate tumor markers, such as polynucleotide expression, can also be used to better classify, and thus diagnose and treat, different forms and disease states of cancer. Two classifications widely used in oncology that can benefit from identification of the expression levels of the genes corresponding to the polynucleotides described herein are staging of the cancerous disorder, and grading the nature of the cancerous tissue.

Measuring TFF3 expression can be useful for monitoring patients having or susceptible to cancer to detect potentially malignant events at a molecular level before they are detectable at a gross morphological level. In addition, TFF3 polynucleotides, as well as the genes corresponding to such polynucleotides, can be useful as therametrics, e.g., to assess the effectiveness of therapy by using the polynucleotides or their encoded gene products, to assess, for example, tumor burden in the patient before, during, and after therapy.

Furthermore, a polynucleotide identified as corresponding to a gene that is differentially expressed in one type of cancer can also have implications for development or risk of development of other types of cancer, e.g., where a polynucleotide represents a gene differentially expressed across various cancer types. Thus, for example, expression of a polynucleotide corresponding to a gene that has clinical implications for metastatic colon cancer can also have clinical implications for stomach cancer or endometrial cancer.

Staging

Staging is a process used by physicians to describe how advanced the cancerous state is in a patient, and staging assists the physician in determining a prognosis, planning treatment and evaluating the results of such treatment. Staging systems vary with the types of cancer, but generally involve the following “TNM” system: the type of tumor, indicated by T; whether the cancer has metastasized to nearby lymph nodes, indicated by N; and whether the cancer has metastasized to more distant parts of the body, indicated by M. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage 1V, the most advanced stage.

The polynucleotides described herein can facilitate fine-tuning of the staging process by identifying markers for the aggressiveness of a cancer, e.g. the metastatic potential, as well as the presence in different areas of the body. Thus, a Stage II cancer with a polynucleotide signifying a high metastatic potential cancer can be used to change a borderline Stage II tumor to a Stage III tumor, justifying more aggressive therapy. Conversely, the presence of a polynucleotide signifying a lower metastatic potential allows more conservative staging of a tumor.

Grading of Cancers

Grade is a term used to describe how closely a tumor resembles normal tissue of its same type. The microscopic appearance of a tumor is used to identify tumor grade based on parameters such as cell morphology, cellular organization, and other markers of differentiation. As a general rule, the grade of a tumor corresponds to its rate of growth or aggressiveness, with undifferentiated or high-grade tumors generally being more aggressive than well differentiated or low-grade tumors. The following guidelines are generally used for grading tumors: 1) GX Grade cannot be assessed; 2) G1 Well differentiated; G2 Moderately well differentiated; 3) G3 Poorly differentiated; 4) G4 Undifferentiated. The polynucleotides contemplated by the invention can be especially valuable in determining the grade of the tumor, as they not only can aid in determining the differentiation status of the cells of a tumor, they can also identify factors other than differentiation that are valuable in determining the aggressiveness of a tumor, such as metastatic potential.

Detection of Cancer

TFF3 expression patterns can be used to detect cancer, particularly colon, breast, and prostate cancer, in a subject. Colorectal cancer is one of the most common neoplasms in humans and perhaps the most frequent form of hereditary neoplasia. Prevention and early detection are key factors in controlling and curing colorectal cancer. Colorectal cancer begins as polyps, which are small, benign growths of cells that form on the inner lining of the colon. Over a period of several years, some of these polyps accumulate additional mutations and become cancerous. Multiple familial colorectal cancer disorders have been identified, which are summarized as follows: 1) Familial adenomatous polyposis (FAP); 2) Gardner's syndrome; 3) Hereditary nonpolyposis colon cancer (HNPCC); and 4) Familial colorectal cancer in Ashkenazi Jews. The expression of appropriate polynucleotides can be used in the diagnosis, prognosis and management of cancer. Detection of cancer can be determined using expression levels of the TFF3 sequence alone or in combination with other genes. Determination of the aggressive nature and/or the metastatic potential of a colon cancer can be determined by comparing levels of one or more gene products of the genes corresponding to the polynucleotides described herein, and comparing total levels of another sequence known to vary in cancerous tissue, e.g., expression of p53, DCC, ras, FAP (see, e.g., Fearon E R, et al., Cell (1990) 61(5):759; Hamilton SR et al., Cancer (1993) 72:957; Bodmer W, et al., Nat Genet. (1994) 4(3):217; Fearon E R, Ann N Y Acad Sci. (1995) 768:101). For example, development of cancer can be detected by examining the level of expression of TFF3 corresponding to polynucleotides described herein to the levels of oncogenes (e.g. ras) or tumor suppressor genes (e.g. FAP or p53). Thus, expression of specific marker polynucleotides can be used to discriminate between normal and cancerous colon tissue, to discriminate between cancers with different cells of origin, to discriminate between cancers with different potential metastatic rates, etc. For a review of markers of cancer, see, e.g., Hanahan et al. (2000) Cell 100:57-70, which in incorporated herein by reference in its entirety.

Treatment of Cancer

The invention provides methods for inhibiting growth of cancer cells and/or modulating the adhesion, migration and/or metastasis of cancers characterized by TFF3 expression. Examples thereof include digestive cancers such as colon cancer, stomach (gastric) cancer and liver cancer, and other cancers, such as lung cancer, breast cancer, ovarian cancer, and prostate cancer. In some embodiments, the cancer shows differential expression of TFF3. In further embodiments, TFF3 is upregulated in the cancer. In yet further embodiments, the cancer is other than colon cancer, and in other embodiments, the cancer is other than prostate cancer.

The invention embraces treatment of any cancer where the administration of a TFF3 neutralizing agent modulates (e.g., inhibits) at least one of cancer cell proliferation, cancer cell migration, cancer cell adhesion and/or metastasis. As shown in the examples, TFF3 neutralizing agents have been demonstrated to inhibit cancer cell adhesion and to inhibit cancer cell proliferation. Inhibition of adhesion can have a modulatory effect on metastasis by inhibiting the ability of a cancer cell to adhere to and develop a tumor at a site different from the original tumor. In general, the methods comprise contacting a cancer cell with a substance that modulates (1) expression of a polynucleotide corresponding to TFF3; or (2) a level of and/or an activity of a TFF3 polypeptide. The methods provide for decreasing the expression of TFF3 in a cancer cell or decreasing the level of and/or decreasing an activity of TFF3. This inhibition can result in decreased cancer cell proliferation, migration and/or adhesion, reduced tumor growth, reduced tumor volume, reduced tumor invasiveness, and the like.

“Reducing growth of tumors or cancer cells” includes, but is not limited to, reducing proliferation of cancer (or tumor) cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring a marker associated with colon cancer (e.g., CEA, CA19-9, and LASA), and the like.

The present invention in particular provides methods for treating TFF3 associated cancer, such as colon cancer, breast cancer, and/or prostate cancer, comprising administering to an individual in need thereof a substance that reduces cancer cell growth, in an amount sufficient to reduce cancer cell growth and treat the cancer. Whether a substance, or a specific amount of the substance, is effective in treating cancer in patients can be assessed using any of a variety of known diagnostic assays for cancer, including, but not limited to, sigmoidoscopy, proctoscopy, rectal examination, colonoscopy with biopsy, contrast radiographic studies, CAT scans, angiography, and detection of a tumor marker associated with colon cancer in the blood of the individual. The substance can be administered systemically or locally. Local administration may be useful in treating, e.g., a solid tumor.

Diagnostic and Other Methods Involving Detection of TFF3

The present invention provides methods of using TFF3 neutralizing agents, TFF3 polypeptides, and TFF3 polynucleotides described herein for diagnostic purposes and other methods. In specific non-limiting embodiments, the methods are useful for detecting TFF3 associated cancer cells, such as colon, breast, ovarian, gastric, and prostate cancer cells, facilitating diagnosis of cancer and the severity of a cancer (e.g., tumor grade, tumor burden, and the like) in a subject, facilitating a determination of the prognosis of a subject, and assessing the responsiveness of the subject to therapy (e.g., by providing a measure of therapeutic effect through, for example, assessing tumor burden during or following a chemotherapeutic regimen). Detection can be based on detection of levels of TFF3 in a cell, e.g., colon cancer cell and/or detection of a TFF3 polypeptide in a cancer cell. The detection methods of the invention can be conducted in vitro or in vivo, on isolated cells, or in whole tissues or a bodily fluid, e.g., blood, plasma, serum, urine, and the like).

Accordingly, the present invention provides methods of detecting TFF3 in a biological sample and detecting the presence of cancer in a biological sample by contacting the sample and detecting evidence of differential expression of TFF3 in the sample. Evidence can be in the form of binding between the TFF3 neutralizing agent and the TFF3 in the sample. Numerous methods for detecting and measuring the level of binding are known in the art and include, for example, ELISA-based assays and the like. Binding levels can be compared against standard samples to indicate if TFF3 expression is lower or higher than in a normal sample. In some embodiments, detection of higher than normal TFF3 expression indicates the presence of cancer.

The present invention further provides methods for determining the susceptibility of a patient to a TFF3 neutralizing agent by detecting evidence of differential expression of TFF3 in a patient's cancer sample. As used herein, the term “susceptible” can describe patients for whom administration of a TFF3 neutralizing agent is an acceptable method of treatment, i.e., the term describes patients who are likely to respond positively to treatment with a TFF3 neutralizing agent. Cancer patients susceptible to the treatment methods of the present invention can exhibit differential expression of TFF3, e.g., in diseased tissue, compared with patients who would not be susceptible to treatment.

The detection methods of the present invention can be provided as part of a kit. Thus, the invention further provides kits for detecting the presence and/or a level of a TFF3 expressed in a cancer cell (e.g., by detection of an mRNA encoded by the differentially expressed gene of interest), and/or a polypeptide encoded thereby, in a biological sample. Procedures using these kits can be performed by clinical laboratories, experimental laboratories, medical practitioners, or private individuals. The kits of the invention for detecting a polypeptide encoded by a polynucleotide that is differentially expressed in a colon cancer cell comprise a moiety that specifically binds the polypeptide, which may be a specific antibody, antisense molecule, RNAi molecule, ribozyme, or small molecule. The kits of the invention for detecting a polynucleotide that is differentially expressed in a cancer cell comprise a moiety that specifically hybridizes to such a polynucleotide. The kit may optionally provide additional components that are useful in the procedure, including, but not limited to, buffers, developing reagents, labels, reacting surfaces, means for detection, control samples, standards, instructions, and interpretive information.

Detecting a TFF3 Polypeptide in a Cancer Cell

In some embodiments, methods are provided for detecting TFF3 associated cancer by detecting an overexpressing TFF3 cell. Any of a variety of known methods can be used for detection, including, but not limited to, immunoassay, using antibody specific for the encoded polypeptide, e.g., by enzyme-linked immunosorbent assay (ELISA), radioimmunoassay (RIA), and the like; and functional assays for the encoded polypeptide, e.g., binding activity or enzymatic activity.

For example, an immunofluorescence assay can be performed on cells without first isolating the encoded polypeptide. The cells are first fixed onto a solid support, such as a microscope slide or microtiter well. This fixing step can permeabilize the cell membrane. The permeablization of the cell membrane permits the polypeptide-specific antibody to bind. Next, the fixed cells are exposed to an antibody specific for the encoded polypeptide. To increase the sensitivity of the assay, the fixed cells can be further exposed to a second antibody, which is labeled and binds to the first antibody, which is specific for the encoded polypeptide. Typically, the secondary antibody is detectably labeled, e.g., with a fluorescent marker. The cells which express the encoded polypeptide will be fluorescently labeled and easily visualized under the microscope. See, for example, Hashido et al. (1992) Biochem. Biophys. Res. Comm. 187:1241-1248.

As will be readily apparent to the ordinarily skilled artisan upon reading the present specification, the detection methods and other methods described herein can be readily varied. Such variations are within the intended scope of the invention. For example, in the above detection scheme, the probe for use in detection can be immobilized on a solid support, and the test sample contacted with the immobilized probe. Binding of the test sample to the probe can then be detected in a variety of ways, e.g., by detecting a detectable label bound to the test sample to facilitate detected of test sample-immobilized probe complexes.

The present invention further provides methods for detecting the presence of and/or measuring a level of TFF3 polypeptide in a biological sample, using an antibody specific for TFF3. The methods generally comprise: a) contacting the sample with an antibody specific for a TFF3; and b) detecting binding between the antibody and molecules of the sample.

Detection of specific binding of the antibody specific for TFF3, when compared to a suitable control, is an indication that TFF3 is present in the sample. Suitable controls include a sample known not to contain TFF3; and a sample contacted with an antibody not specific for the encoded polypeptide, e.g., an anti-idiotype antibody. A variety of methods to detect specific antibody-antigen interactions are known in the art and can be used in the method, including, but not limited to, standard immunohistological methods, immunoprecipitation, an enzyme immunoassay, and a radioimmunoassay. In general, the specific antibody will be detectably labeled, either directly or indirectly. Direct labels include radioisotopes; enzymes whose products are detectable (e.g., luciferase, galactosidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin, aequorin (green fluorescent protein), and the like. The antibody can be attached (coupled) to an insoluble support, such as a polystyrene plate or a bead. Indirect labels include second antibodies specific for antibodies specific for the encoded polypeptide (“first specific antibody”), wherein the second antibody is labeled as described above; and members of specific binding pairs, e.g., biotin-avidin, and the like. The biological sample may be brought into contact with and immobilized on a solid support or carrier, such as nitrocellulose, that is capable of immobilizing cells, cell particles, or soluble proteins. The support may then be washed with suitable buffers, followed by contacting with a detectably-labeled first specific antibody. Detection methods are known in the art and will be chosen as appropriate to the signal emitted by the detectable label. Detection is generally accomplished in comparison to suitable controls, and to appropriate standards.

In some embodiments, the methods are adapted for use in vivo, e.g., to locate or identify sites where TFF3 associated cancer cells are present. In these embodiments, a detectably-labeled moiety, e.g., an antibody, which is specific for TFF3 administered to an individual (e.g., by injection), and labeled cells are located using standard imaging techniques, including, but not limited to, magnetic resonance imaging, computed tomography scanning, and the like. In this manner, TFF3 expressing cells are differentially labeled.

Detecting a TFF3 Polynucleotide in a Cancer Cell

Methods are provided for detecting a TFF3 cancer cell by detecting expression in the cell of a TFF3 transcript in a cancer cell. Any of a variety of known methods can be used for detection, including, but not limited to, detection of a transcript by hybridization with a polynucleotide that hybridizes to a TFF3 polynucleotide; detection of a transcript by a polymerase chain reaction using specific oligonucleotide primers; in situ hybridization of a cell using as a probe a polynucleotide that hybridizes to a gene that is differentially expressed in a colon cancer cell. The methods can be used to detect and/or measure mRNA levels TFF3 gene expressed in a cancer cell. In some embodiments, the methods comprise: a) contacting a sample with a TFF3 polynucleotide under conditions that allow hybridization; and b) detecting hybridization, if any.

Detection of differential hybridization, when compared to a suitable control, is an indication of the presence in the sample of a polynucleotide that is differentially expressed in a cancer cell. Appropriate controls include, for example, a sample which is known not to contain a TFF3 polynucleotide. Conditions that allow hybridization are known in the art. Detection can also be accomplished by any known method, including, but not limited to, in situ hybridization, PCR (polymerase chain reaction), RT-PCR (reverse transcription-PCR), and “Northern” or RNA blotting, or combinations of such techniques, using a suitably labeled polynucleotide. A variety of labels and labeling methods for polynucleotides are known in the art and can be used in the assay methods of the invention. Specific hybridization can be determined by comparison to appropriate controls.

Polynucleotides generally comprising at least 12 contiguous nt of the TFF3 polynucleotides provided herein, can be used for a variety of purposes, such as probes for detection of and/or measurement of, transcription levels of a polynucleotide that is differentially expressed in a colon cancer cell. A probe that hybridizes specifically to a polynucleotide disclosed herein should provide a detection signal at least 5-, 10-, or 20-fold higher than the background hybridization provided with other unrelated sequences. It should be noted that “probe” as used herein is meant to refer to a polynucleotide sequence used to detect a TFF3 gene product in a test sample. As will be readily appreciated by the ordinarily skilled artisan, the probe can be detectably labeled and contacted with, for example, an array comprising immobilized polynucleotides obtained from a test sample (e.g., mRNA). Alternatively, the probe can be immobilized on an array and the test sample detectably labeled. These and other variations of the methods of the invention are well within the skill in the art and are within the scope of the invention.

Nucleotide probes can be used to detect expression of a gene corresponding to the provided polynucleotide. In Northern blots, mRNA is separated electrophoretically and contacted with a probe. A probe is detected as hybridizing to an mRNA species of a particular size. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes are typically labeled with a radioactive isotope. Other types of detectable labels can be used such as chromophores, fluorophores, and enzymes. Other examples of nucleotide hybridization assays are described in WO92/02526 and U.S. Pat. No. 5,124,246.

PCR is another means for detecting small amounts of target nucleic acids (see, e.g., Mullis et al., Meth. Enzymol. (1987) 155:335; U.S. Pat. Nos. 4,683,195 and 4,683,202). Two primer polynucleotides nucleotides that hybridize with the target nucleic acids are used to prime the reaction. The primers can comprise a sequence within or 3′ and 5′ to the polynucleotides disclosed herein. Alternatively, if the primers are 3′ and 5′ to these polynucleotides, they need not hybridize to them or the complements. After amplification of the target with a thermostable polymerase, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase enzyme can be purified and separated using gel electrophoresis, and transferred to a solid support, such as nitrocellulose. The solid support is exposed to a labeled probe, washed to remove any unhybridized probe, and duplexes containing the labeled probe are detected.

Methods using PCR amplification can be performed on the DNA from a single cell, although it is convenient to use at least about 10⁵ cells. The use of the polymerase chain reaction is described in Saiki et al. (1985) Science 239:487, and a review of current techniques may be found in Sambrook, et al. Molecular Cloning: A Laboratory Manual, CSH Press 1989, pp. 14.2-14.33, each of which is incorporated herein by reference. A detectable label can be included in the amplification reaction. Suitable detectable labels include fluorochromes,(e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA)), radioactive labels, (e.g. ³²P, ³⁵S, ³H, etc.), and the like. The label may be a two stage system, where the polynucleotides is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

Arrays

Polynucleotide arrays provide a high throughput technique that can assay a large number of polynucleotides or polypeptides in a sample. This technology can be used as a tool to test for differential expression. A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions. Samples of polynucleotides can be detectably labeled (e.g., using radioactive or fluorescent labels) and then hybridized to the probes. Double stranded polynucleotides, comprising the labeled sample polynucleotides bound to probe polynucleotides, can be detected once the unbound portion of the sample is washed away. Alternatively, the polynucleotides of the test sample can be immobilized on the array, and the probes detectably labeled. Techniques for constructing arrays and methods of using these arrays are described in, for example, Schena et al. (1996) Proc Natl Acad Sci USA. 93(20):10614-9; Schena et al. (1995) Science 270(5235):467-70; Shalon et al. (1996) Genome Res. 6(7):639-45, U.S. Pat. No. 5,807,522, EP 799 897; WO 97/29212; WO 97/27317; EP 785 280; WO 97/02357; U.S. Pat. No. 5,593,839; U.S. Pat. No. 5,578,832; EP 728 520; U.S. Pat. No. 5,599,695; EP 721 016; U.S. Pat. No. 5,556,752; WO 95/22058; and U.S. Pat. No. 5,631,734.

Arrays can be used to, for example, examine differential expression of genes and can be used to determine gene function. For example, arrays can be used to detect differential expression of a TFF3 gene, where expression is compared between a test cell and control cell (e.g., cancer cells and normal cells). For example, high expression of a particular message in a cancer cell, which is not observed in a corresponding normal cell, can indicate a cancer specific gene product. Exemplary uses of arrays are further described in, for example, Pappalarado et al., Sem. Radiation Oncol. (1998) 8:217; and Ramsay Nature Biotechnol. (1998) 16:40. Furthermore, many variations on methods of detection using arrays are well within the skill in the art and within the scope of the present invention. For example, rather than immobilizing the probe to a solid support, the test sample can be immobilized on a solid support which is then contacted with the probe.

Articles of Manufacture

In other embodiments of the invention, an article of manufacture containing a TFF3 neutralizing agent useful for the treatment of the diseases or disorders described herein is provided. The article of manufacture comprises a container and a label or package insert on or associated with the container. Suitable containers include, for example, bottles, vials, syringes, etc. The containers can be formed from a variety of materials such as glass or plastic. The container holds or contains a composition that is effective for treating the disease or disorder of choice and may have a sterile access port (for example the container may be an intravenous solution bag or a vial having a stopper pierceable by a hypodermic injection needle). At least one active agent in the composition is a TFF3 neutralizing agent, such as an TFF3 antisense molecule, RNAi molecule, ribozyme, small molecule, or antibody. The label or package insert indicates that the composition is used for treating a patient having or predisposed to cancer, e.g., colon, breast or prostate cancer. The article of manufacture can further include a second container having a pharmaceutically acceptable diluent buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Further details of the invention are illustrated by the following non-limiting Examples. The disclosures of all citations in the specification are expressly incorporated herein by reference in their entireties.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in ° C., and pressure is at or near atmospheric.

### Example 1

### Source of Biological Materials

The biological materials used in the experiments that led to the present invention are described below.

Source of Patient Tissue Samples

Normal and cancerous tissues were collected from patients using laser capture microdissection (LCM) techniques, which techniques are well known in the art (see, e.g., Ohyama et al. (2000) Biotechniques 29:530-6; Curran et al. (2000) Mol. Pathol. 53:64-8; Suarez-Quian et al. (1999) Biotechniques 26:328-35; Simone et al. (1998) Trends Genet 14:272-6; Conia et al. (1997) J. Clin. Lab. Anal. 11:28-38; Emmert-Buck et al. (1996) Science 274:998-1001). Table 2 below provides information about each patient from which the prostate tissue samples were isolated, including: 1) the “Patient ID”, which is a number assigned to the patient for identification purposes; 2) the “Tissue Type”; and 3) the “Gleason Grade” of the tumor. Histopathology of all primary tumors indicated the tumor was adenocarcinoma.

TABLE 2
Prostate patient data.

| Patient | | Gleason | Patient | | Gleason |
|---|---|---|---|---|---|
| ID | Tissue Type | Grade | ID | Tissue Type | Grade |
| 93 | Prostate Cancer | 3 + 4 | 391 | Prostate Cancer | 3 + 3 |
| 94 | Prostate Cancer | 3 + 3 | 420 | Prostate Cancer | 3 + 3 |
| 95 | Prostate Cancer | 3 + 3 | 425 | Prostate Cancer | 3 + 3 |
| 96 | Prostate Cancer | 3 + 3 | 428 | Prostate Cancer | 4 + 3 |
| 97 | Prostate Cancer | 3 + 2 | 431 | Prostate Cancer | 3 + 4 |
| 100 | Prostate Cancer | 3 + 3 | 492 | Prostate Cancer | 3 + 3 |
| 101 | Prostate Cancer | 3 + 3 | 493 | Prostate Cancer | 3 + 4 |
| 104 | Prostate Cancer | 3 + 3 | 496 | Prostate Cancer | 3 + 3 |
| 105 | Prostate Cancer | 3 + 4 | 510 | Prostate Cancer | 3 + 3 |
| 106 | Prostate Cancer | 3 + 3 | 511 | Prostate Cancer | 4 + 3 |
| 138 | Prostate Cancer | 3 + 3 | 514 | Prostate Cancer | 3 + 3 |
| 151 | Prostate Cancer | 3 + 3 | 549 | Prostate Cancer | 3 + 3 |
| 153 | Prostate Cancer | 3 + 3 | 552 | Prostate Cancer | 3 + 3 |
| 155 | Prostate Cancer | 4 + 3 | 858 | Prostate Cancer | 3 + 4 |
| 171 | Prostate Cancer | 3 + 4 | 859 | Prostate Cancer | 3 + 4 |
| 173 | Prostate Cancer | 3 + 4 | 864 | Prostate Cancer | 3 + 4 |
| 231 | Prostate Cancer | 3 + 4 | 883 | Prostate Cancer | 4 + 4 |
| 232 | Prostate Cancer | 3 + 3 | 895 | Prostate Cancer | 3 + 3 |
| 251 | Prostate Cancer | 3 + 4 | 901 | Prostate Cancer | 3 + 3 |
| 282 | Prostate Cancer | 4 + 3 | 909 | Prostate Cancer | 3 + 3 |
| 286 | Prostate Cancer | 3 + 3 | 921 | Prostate Cancer | 3 + 3 |
| 294 | Prostate Cancer | 3 + 4 | 923 | Prostate Cancer | 4 + 3 |
| 351 | Prostate Cancer | 5 + 4 | 934 | Prostate Cancer | 3 + 3 |
| 361 | Prostate Cancer | 3 + 3 | 1134 | Prostate Cancer | 3 + 4 |
| 362 | Prostate Cancer | 3 + 3 | 1135 | Prostate Cancer | 3 + 3 |
| 365 | Prostate Cancer | 3 + 2 | 1136 | Prostate Cancer | 3 + 4 |
| 368 | Prostate Cancer | 3 + 3 | 1137 | Prostate Cancer | 3 + 3 |
| 379 | Prostate Cancer | 3 + 4 | 1138 | Prostate Cancer | 4 + 3 |
| 388 | Prostate Cancer | 5 + 3 | | | |

### Example 2

### Detection of Differential Expression Using Arrays

cDNA probes were prepared from total RNA isolated from the patient cells described above in Example 1. Since LCM provides for the isolation of specific cell types to provide a substantially homogenous cell sample, this provided for a similarly pure RNA sample.

Total RNA was first reverse transcribed into cDNA using a primer containing a T7 RNA polymerase promoter, followed by second strand DNA synthesis. cDNA was then transcribed in vitro to produce antisense RNA using the T7 promoter-mediated expression (see, e.g., Luo et al. (1999) Nature Med 5:117-122), and the antisense RNA was then converted into cDNA. The second set of cDNAs were again transcribed in vitro, using the T7 promoter, to provide antisense RNA. Optionally, the RNA was again converted into cDNA, allowing for up to a third round of T7-mediated amplification to produce more antisense RNA. Thus the procedure provided for two or three rounds of in vitro transcription to produce the final RNA used for fluorescent labeling.

Fluorescent probes were generated by first adding control RNA to the antisense RNA mix, and producing fluorescently labeled cDNA from the RNA starting material. Fluorescently labeled cDNAs prepared from the tumor RNA sample were compared to fluorescently labeled cDNAs prepared from normal cell RNA sample. For example, the cDNA probes from the normal cells were labeled with Cy3 fluorescent dye (green) and the cDNA probes prepared from the tumor cells were labeled with Cy5 fluorescent dye (red), and vice versa.

Each array used had an identical spatial layout and control spot set. Each microarray was divided into two areas, each area having an array with, on each half, twelve groupings of 32×12 spots, for a total of about 9,216 spots on each array. The two areas are spotted identically which provide for at least two duplicates of each clone per array.

Polynucleotides for use on the arrays were obtained from both publicly available sources and from cDNA libraries generated from selected cell lines and patient tissues as described above. PCR products of from about 0.5 kb to 2.0 kb amplified from these sources were spotted onto the array using a Molecular Dynamics Gen III spotter according to the manufacturer's recommendations. The first row of each of the 24 regions on the array had about 32 control spots, including 4 negative control spots and 8 test polynucleotides. The test polynucleotides were spiked into each sample before the labeling reaction with a range of concentrations from 2-600 pg/slide and ratios of 1:1. For each array design, two slides were hybridized with the test samples reverse-labeled in the labeling reaction. This provided for about four duplicate measurements for each clone, two of one color and two of the other, for each sample.

The differential expression assay was performed by mixing equal amounts of probes from tumor cells and normal cells of the same patient. The arrays were prehybridized by incubation for about 2 hrs at 60° C. in 5×SSC/0.2% SDS/1 mM EDTA, and then washed three times in water and twice in isopropanol. Following prehybridization of the array, the probe mixture was then hybridized to the array under conditions of high stringency (overnight at 42° C. in 50% formamide, 5×SSC, and 0.2% SDS. After hybridization, the array was washed at 55° C. three times as follows: 1) first wash in 1×SSC/0.2% SDS; 2) second wash in 0.1×SSC/0.2% SDS; and 3) third wash in 0.1× ssc.

The arrays were then scanned for green and red fluorescence using a Molecular Dynamics Generation III dual color laser-scanner/detector. The images were processed using BioDiscovery Autogene software, and the data from each scan set normalized to provide for a ratio of expression relative to normal. Data from the microarray experiments was analyzed according to the algorithms described in U.S. application Ser. No. 60/252,358, filed Nov. 20, 2000, by E. J. Moler, M. A. Boyle, and F. M. Randazzo, and entitled “Precision and accuracy in cDNA microarray data,” which application is specifically incorporated herein by reference.

The experiment was repeated, this time labeling the two probes with the opposite color in order to perform the assay in both “color directions.” Each experiment was sometimes repeated with two more slides (one in each color direction). The level fluorescence for each sequence on the array expressed as a ratio of the geometric mean of 8 replicate spots/genes from the four arrays or 4 replicate spots/gene from 2 arrays or some other permutation. The data were normalized using the spiked positive controls present in each duplicated area, and the precision of this normalization was included in the final determination of the significance of each differential. The fluorescent intensity of each spot was also compared to the negative controls in each duplicated area to determine which spots have detected significant expression levels in each sample.

A statistical analysis of the fluorescent intensities was applied to each set of duplicate spots to assess the precision and significance of each differential measurement, resulting in a p-value testing the null hypothesis that there is no differential in the expression level between the tumor and normal samples of each patient. During initial analysis of the microarrays, the hypothesis was accepted if p>10⁻³, and the differential ratio was set to 1.000 for those spots. All other spots have a significant difference in expression between the tumor and normal sample. If the tumor sample has detectable expression and the normal does not, the ratio is truncated at 1000 since the value for expression in the normal sample would be zero, and the ratio would not be a mathematically useful value (e.g., infinity). If the normal sample has detectable expression and the tumor does not, the ratio is truncated to 0.001, since the value for expression in the tumor sample would be zero and the ratio would not be a mathematically useful value. These latter two situations are referred to herein as “on/off.”

### Example 3

### Expression of TFF3 in Cells

TFF3 mRNA Upregulation in Cancer

Cancer cells and adjacent normal cells from tumor samples were collected by Laser-Capture Micro-Dissection from each cancer patient sample (see Example 1 for source of tissue samples). Labeled probes were prepared from the RNA of each sample and used to probe cDNA microarray chips (see Example 2). Each microarray chip was hybridized simultaneously with the normal and cancer probes for an individual patient (normal and cancer were labeled with different fluorescent reagents). The expression level was determined as a ratio of the expression in cancer over the expression in normal cells for each patient sample. Table 3 indicates the percentage of patients in which the expression ratio in cancer over the normal epithelial cells is higher than 2-fold (>2×), higher than 5-fold (>5×) or lower than one half (<0.5×), in at least 20% of patients tested.

TABLE 3
TFF3 mRNA Up-Regulation in Cancer

| Cancer | # patients | >2× | >5× | <0.5× |
|---|---|---|---|---|
| Prostate | 102 | ˜40% | ˜27% | ˜15% |
| Breast | 23 | ˜40% | ˜27% | ˜30% |
| Colon | 77 | ˜20% | ˜6% | ˜30% |
| Colon Mets | 33 | ˜22% | ˜2% | ˜20% |

Normal Tissue Expression of TFF3

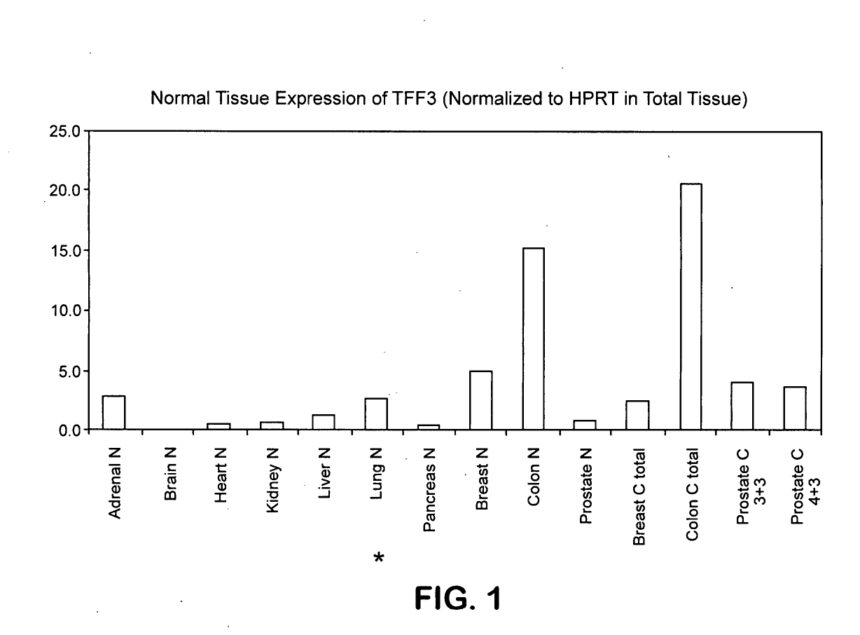

Expression of TFF3 mRNA in whole tissue (e.g., not Laser-Capture Dissected samples, thus representing all cell types in the tissue sample), both normal (N) and cancer (C), was determined by real-time quantitative PCR. The results are shown in FIG. 1 and values are normalized to HPRT in Total Tissue. The values for the level of mRNA expression on the y-axis are relative numbers using HPRT as the normalization control. The cancer samples represent pools of tumor samples from 8 individual patients (see Example 1 for source of biological materials). The nomenclature “3+3” refers to Gleason grade 6 and “4+3” refers to Gleason grade 7. It is also noted that in normal lung (as in colon), TFF3 is expressed by goblet cells and secreted with mucous (see, e.g., Am. J. Respir. Crit. Care Med., 1999, 159, 1330).

Immunohistochemical (IHC) Detection of TFF3 in Cancer Patients

Tissue sample sections (Total # of Tissues) for individual breast, colon, ovarian, and prostate cancer (CA) patients and normal tissue counterparts (NL) were stained with immunoaffinity purified rabbit polyclonal antibodies to human TFF3. In addition, samples for several vital normal organs were stained. The number of samples that were negative (−) and positive (+) for the presence of TFF3 are provided in Table 4, as well as the percentage of TFF3 positive samples in each category.

TABLE 4
IHC Summary

| Tissue | Total # of | (−) | (+) | % |
|---|---|---|---|---|
| Categorey | Tissues | Samples | Samples | Positives |
| Breast CA | 77 | 21 | 56 | 72.7% |
| Breast NL | 20 | 15 | 5 | 25.0% |
| Colon CA | 45 | 16 | 29 | 64.4% |
| Colon NL | 32 | 1 | 31 | 96.9% |
| Ovary CA | 31 | 25 | 6 | 19.4% |
| Ovary NL | 25 | 25 | 0 | 0.0% |
| Prostate CA | 67 | 24 | 43 | 64.2% |
| Prostate NL | 18 | 17 | 1 | 5.6% |
| Adrenal NL | 4 | 0 | 4 | 100.0% |
| Brain NL | 8 | 8 | 0 | 0.0% |
| Heart NL | 10 | 10 | 0 | 0.0% |
| Kidney NL | 7 | 6 | 1 | 14.3% |
| Liver NL | 8 | 8 | 0 | 0.0% |
| Pancreas NL | 7 | 7 | 0 | 0.0% |

Expression of TFF3 in Cell Lines

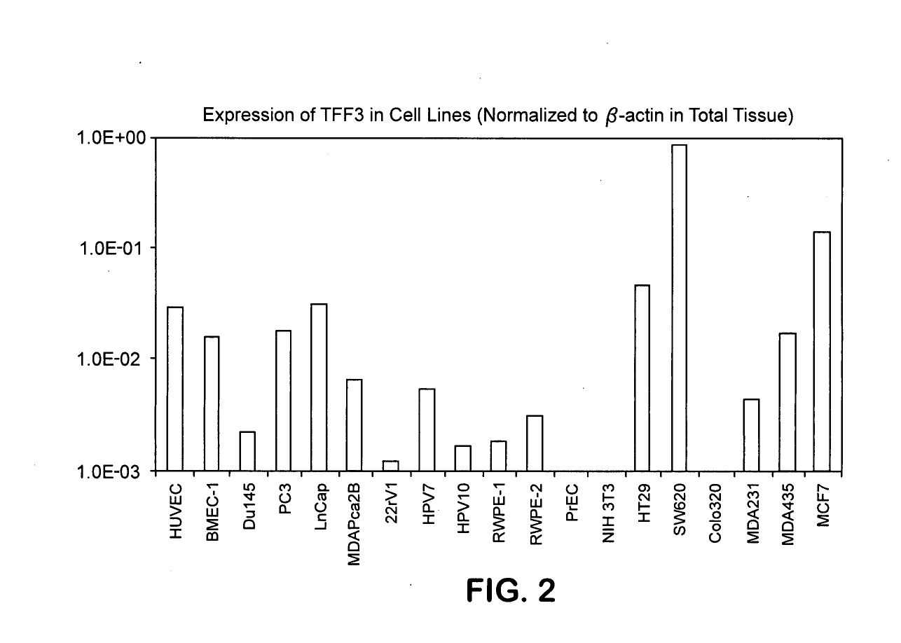

Expression of TFF3 mRNA in certain cell lines was determined by real-time quantitative PCR. Results are provided in FIG. 2. The values for the level of expression are relative numbers using β-actin as the normalization control. Note also that the y-axis is a logarithmic scale, indicating that TFF3 expression varied in cell lines by very large margins. Cell lines included HUVEC (Human Umbilical Vein Endothelial Cells), BMEC-1 (brain microvascular endothelial cells), Du145 (human prostate carcinoma cells), PC3 (human prostatic carcinoma cells), LnCap (human prostate carcinoma cells), MDAPca2B (human prostate carcinoma cells), 22rV1 (human prostate carcinoma cells), HPV7 (human prostate carcinoma cells), HPV10 (human prostate carcinoma cells), RWPE-1 (human prostate epithelial cells), RWPE-2 (malignant human prostate epithelial cells), PrEC (human prostate epithelial cells), NIH 3T3 (fibroblasts), HT29 (human colon carcinoma), SW620 (human colon carcinoma), Colo320 (human colon carcinoma), MDA231 (breast cancer), MDA435 (breast cancer), and MCF7 (breast cancer).

### Example 4

### Antisense Regulation of Gene Expression

The expression of the differentially expressed genes represented by the polynucleotides in cancerous cells can be analyzed using antisense knockout technology to confirm the role and function of the gene product in tumorigenesis, e.g., in promoting a metastatic phenotype.

A number of different oligonucleotides complementary to the mRNA generated by the differentially expressed genes identified herein can be designed as potential antisense oligonucleotides, and tested for their ability to suppress expression of the genes. Sets of antisense oligomers specific to each candidate target are designed using the sequences of the polynucleotides corresponding to a differentially expressed gene and the software program HYBsimulator Version 4 (available for Windows 95/Windows NT or for Power Macintosh, RNAture, Inc. 1003 Health Sciences Road, West, Irvine, Calif. 92612 USA). Factors that are considered when designing antisense oligonucleotides include: 1) the secondary structure of oligonucleotides; 2) the secondary structure of the target gene; 3) the specificity with no or minimum cross-hybridization to other expressed genes; 4) stability; 5) length and 6) terminal GC content. The antisense oligonucleotide is designed so that it will hybridize to its target sequence under conditions of high stringency at physiological temperatures (e.g., an optimal temperature for the cells in culture to provide for hybridization in the cell, e.g., about 37° C.), but with minimal formation of homodimers.

Using the sets of oligomers and the HYBsimulator program, three to ten antisense oligonucleotides and their reverse controls are designed and synthesized for each candidate mRNA transcript, which transcript is obtained from the gene corresponding to the target polynucleotide sequence of interest. Once synthesized and quantitated, the oligomers are screened for efficiency of a transcript knock-out in a panel of cancer cell lines. The efficiency of the knock-out is determined by analyzing mRNA levels using lightcycler quantification. The oligomers that resulted in the highest level of transcript knock-out, wherein the level was at least about 50%, preferably about 80-90%, up to 95% or more up to undetectable message, are selected for use in a cell-based proliferation assay, an anchorage independent growth assay, and an apoptosis assay.

The ability of each designed antisense oligonucleotide to inhibit gene expression can be tested through transfection into LNCaP, PC3, 22Rv1, MDA-PCA-2b, or DU145 prostate carcinoma cells. For each transfection mixture, a carrier molecule (such as a lipid, lipid derivative, lipid-like molecule, cholesterol, cholesterol derivative, or cholesterol-like molecule) is prepared to a working concentration of 0.5 mM in water, sonicated to yield a uniform solution, and filtered through a 0.45 μm PVDF membrane. The antisense or control oligonucleotide is then prepared to a working concentration of 100 μM in sterile Millipore water. The oligonucleotide is further diluted in OptiMEM™ (Gibco/BRL), in a microfuge tube, to 2 μM, or approximately 20 μg oligo/ml of OptiMEM™. In a separate microfuge tube, the carrier molecule, typically in the amount of about 1.5-2 nmol carrier/μg antisense oligonucleotide, is diluted into the same volume of OptiMEM™ used to dilute the oligonucleotide. The diluted antisense oligonucleotide is immediately added to the diluted carrier and mixed by pipetting up and down. Oligonucleotide is added to the cells to a final concentration of 30 nM.

The level of target mRNA that corresponds to a target gene of interest in the transfected cells is quantitated in the cancer cell lines using the Roche LightCycler™ real-time PCR machine or PerkinElmer GeneAmp machine. Values for the target mRNA are normalized versus an internal control (e.g., beta-actin). For each 20 μl reaction, extracted RNA (generally 0.2-1 μg total) is placed into a sterile 0.5 or 1.5 ml microcentrifuge tube, and water is added to a total volume of 12.5 μl. To each tube is added 7.5 μl of a buffer/enzyme mixture, prepared by mixing (in the order listed) 2.5 μl H₂O, 2.0 μl 10× reaction buffer, 10 μl oligo dT (20 pmol), 1.0 μl dNTP mix (10 mM each), 0.5 μl RNAsin®) (20u) (Ambion, Inc., Hialeah, FL), and 0.5 μl MMLV reverse transcriptase (50u) (Ambion, Inc.). The contents are mixed by pipetting up and down, and the reaction mixture is incubated at 42° C. for 1 hour. The contents of each tube are centrifuged prior to amplification.

An amplification mixture is prepared by mixing in the following order: 1×PCR buffer II, 3 mM MgCl₂, 140 μM each dNTP, 0.175 pmol each oligo, 1:50,000 dil of SYBR® Green, 0.25 mg/ml BSA, 1 unit Taq polymerase, and H₂O to 20 μl. (PCR buffer II is available in 10× concentration from Perkin-Elmer, Norwalk, Conn.). In 1× concentration it contains 10 mM Tris pH 8.3 and 50 mM KCl. SYBR® Green (Molecular Probes, Eugene, Oreg.) is a dye which fluoresces when bound to double stranded DNA. As double stranded PCR product is produced during amplification, the fluorescence from SYBR®) Green increases. To each 20 μl aliquot of amplification mixture, 2 PI of template RT is added, and amplification is carried out according to standard protocols. The results are expressed as the percent decrease in expression of the corresponding gene product relative to non-transfected cells, vehicle-only transfected (mock-transfected) cells, or cells transfected with reverse control oligonucleotides.

### Example 5

### Antisense Modulation of TFF3 Expression

Antisense Oligonucleotides

Several antisense (AS) oligonucleotides were designed, prepared, and tested for their ability to modulate TFF3 mRNA levels in SW620 cells (a colon cancer cell line that expresses high levels of TFF3) according to the procedures set out in Example 4. The oligonucleotides are provided in Table 1, supra. Antisense oligonucleotides with a relatively high degree of knockdown activity included those having SEQ ID NOS: 9, 10, 15, 16, 17 and 18. For each of these, an oligonucleotide with the reverse sequence was synthesized for use as a control (RC) to show antisense specificity.

Knockdown of TFF3 mRNA Using AS

The effectiveness of the TFF3 antisense oligonucleotides was tested by transfecting each oligonucleotide into SW620 cells and measuring the remaining TFF3 mRNA levels at about 36 hours post-transfection, using real-time quantitative PCR.

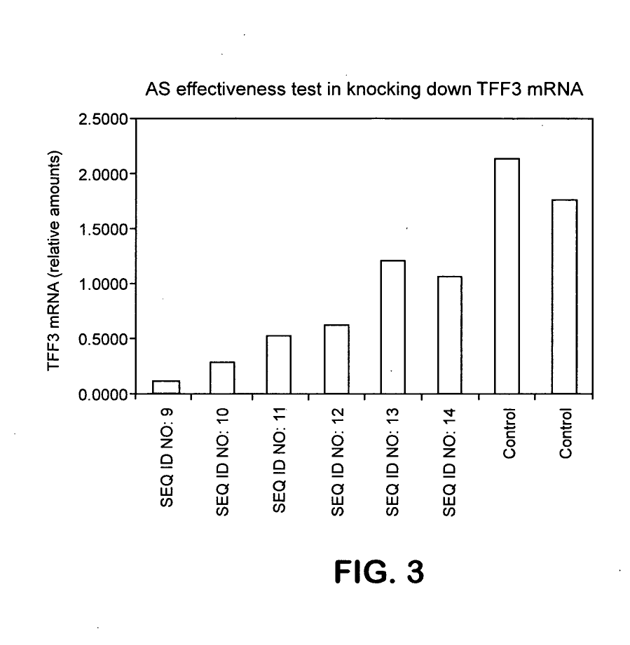

FIG. 3 depicts the results of an initial screening of AS oligonucleotides corresponding to SEQ ID NOS: 9-18. AS oligonucleotides corresponding to SEQ ID NOS: 9 and 10 appeared to be the most effective. AS oligonucleotides designated as Control 1 and Control 2 represent irrelevant AS sequences and serve as controls to assess specific knockdown of TFF3 mRNA.

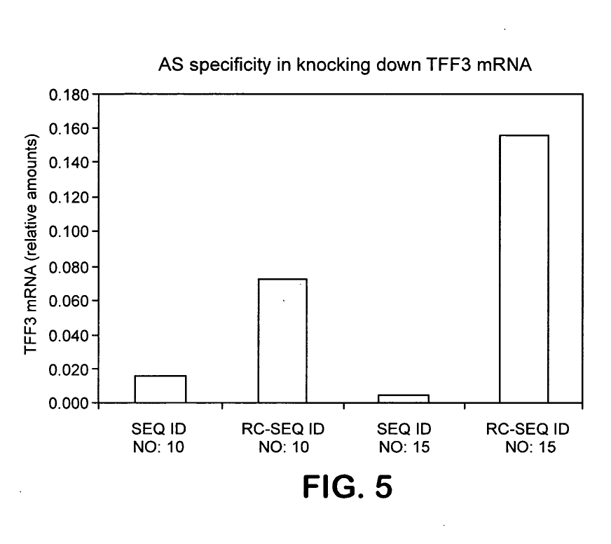

FIGS. 4 and 5 depict the results of a comparison of AS oligonucleotides corresponding to SEQ ID NOS: 9, 10 and 15 versus their reverse controls (RC). As can be seen, the AS oligonucleotides appeared to be acting with desired specificity.

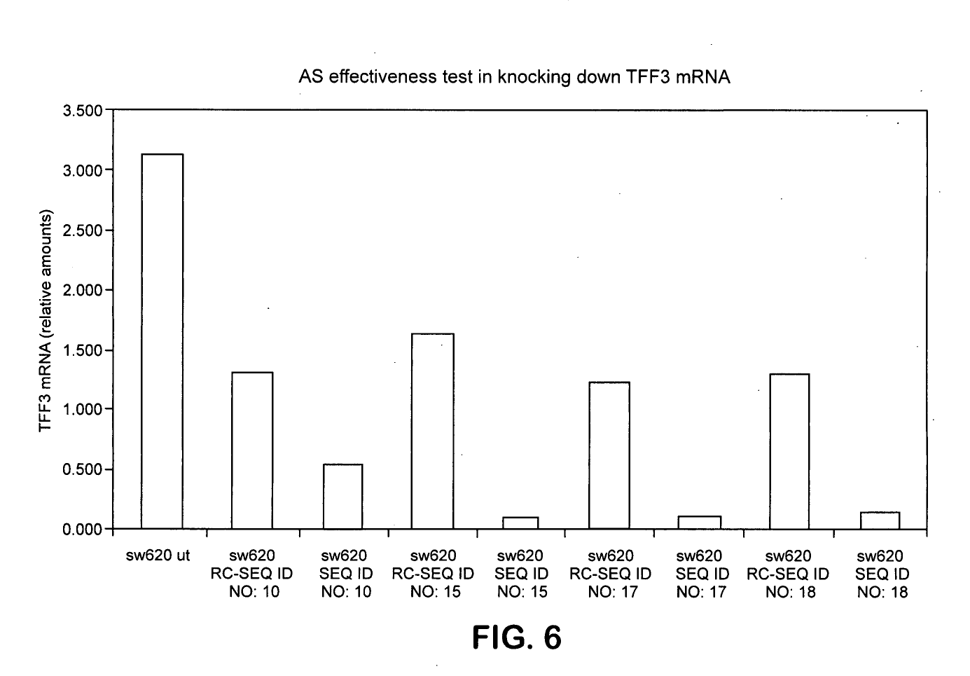

FIG. 6 further depicts the effectiveness of AS oligonucleotides corresponding to SEQ ID NOS: 10, 15, 17 and 18 in knockdown of TFF3 mRNA expression. The term “UT” denotes untransfected SW620.

### Example 6

### Effect of Expression on Proliferation

The effect of gene expression on the inhibition of cell proliferation can be assessed in metastatic breast cancer cell lines (MDA-MB-231 (“231”)); SW620 colon colorectal carcinoma cells; SKOV3 cells (a human ovarian carcinoma cell line); or LNCaP, PC3, 22Rv1, MDA-PCA-2b, or DU145 prostate cancer cells.

Cells are plated to approximately 60-80% confluency in 96-well dishes. Antisense or reverse control oligonucleotide is diluted to 2 μM in OptiMEM™. The oligonucleotide-OptiMEM™ can then be added to a delivery vehicle, which delivery vehicle can be selected so as to be optimized for the particular cell type to be used in the assay. The oligo/delivery vehicle mixture is then further diluted into medium with serum on the cells. The final concentration of oligonucleotide for all experiments can be about 300 nM.

Antisense oligonucleotides are prepared as described above (see Examples 4 and 5). Cells are transfected overnight at 37° C. and the transfection mixture is replaced with fresh medium the next morning. Transfection is carried out as described above in Examples 4 and 5.

Those antisense oligonucleotides that result in inhibition of proliferation of SW620 cells indicate that the corresponding gene plays a role in production or maintenance of the cancerous phenotype in cancerous colon cells. Those antisense oligonucleotides that inhibit proliferation in SKOV3 cells represent genes that play a role in production or maintenance of the cancerous phenotype in cancerous breast cells. Those antisense oligonucleotides that result in inhibition of proliferation of MDA-MB-231 cells indicate that the corresponding gene plays a role in production or maintenance of the cancerous phenotype in cancerous ovarian cells. Those antisense oligonucleotides that inhibit proliferation in LNCaP, PC3, 22Rv1, MDA-PCA-2b, or DU145 cells represent genes that play a role in production or maintenance of the cancerous phenotype in cancerous prostate cells.

### Example 7

### Induction of Cell Death upon Depletion of Polypeptides by Depletion of mRNA

In order to assess the effect of depletion of a target message upon cell death, LNCaP, PC3, 22Rv1, MDA-PCA-2b, or DU145 cells, or other cells derived from a cancer of interest, can be transfected for proliferation assays. For cytotoxic effect in the presence of cisplatin (cis), the same protocol is followed but cells are left in the presence of 2 μM drug. Each day, cytotoxicity is monitored by measuring the amount of LDH enzyme released in the medium due to membrane damage. The activity of LDH is measured using the Cytotoxicity Detection Kit from Roche Molecular Biochemicals. The data is provided as a ratio of LDH released in the medium vs. the total LDH present in the well at the same time point and treatment (rLDH/tLDH). A positive control using antisense and reverse control oligonucleotides for BCL2 (a known anti-apoptotic gene) is included; loss of message for BCL2 leads to an increase in cell death compared with treatment with the control oligonucleotide (background cytotoxicity due to transfection).

### Example 8

### Cytotoxic and Anti-Proliferative Activity of TFF3 Antisense Effects in Cancer cells

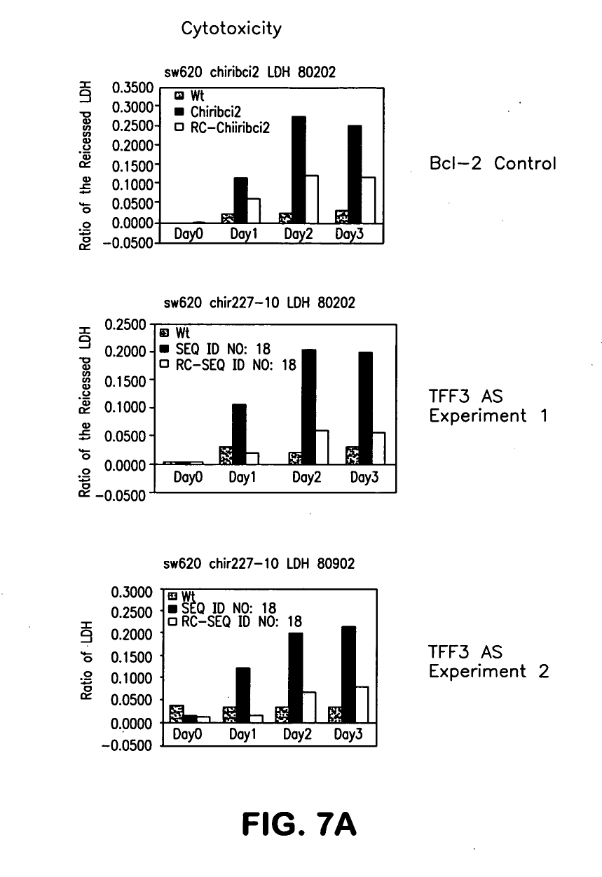

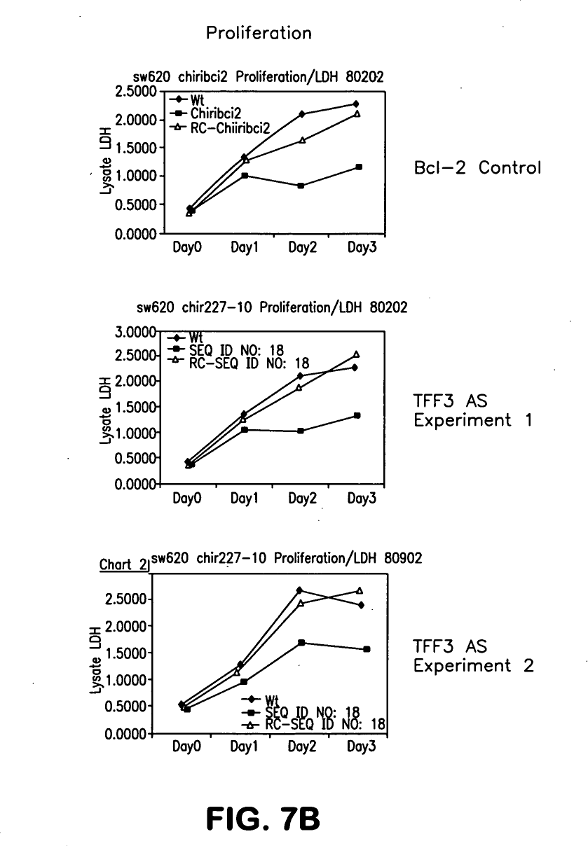

Cytotoxic and anti-proliferative effects of TFF3 antisense oligonucleotides were tested in SW620 cells according to the procedures of Examples 6 and 7 at different time points after transfection of AS and RC oligonucleotides (Examples 4 and 5). Cytotoxicity was determined by measuring the ratio of LDH (lactate dehydrogenase) released into the culture media over total LDH. Proliferation was indicated by the LDH levels in intact adherent cells. As a positive control, Bcl-2 specific antisense was also transfected. Bcl-2 is a known anti-apoptotic protein and blocked expression of this gene results in increased apoptosis. The results are shown in FIG. 7.

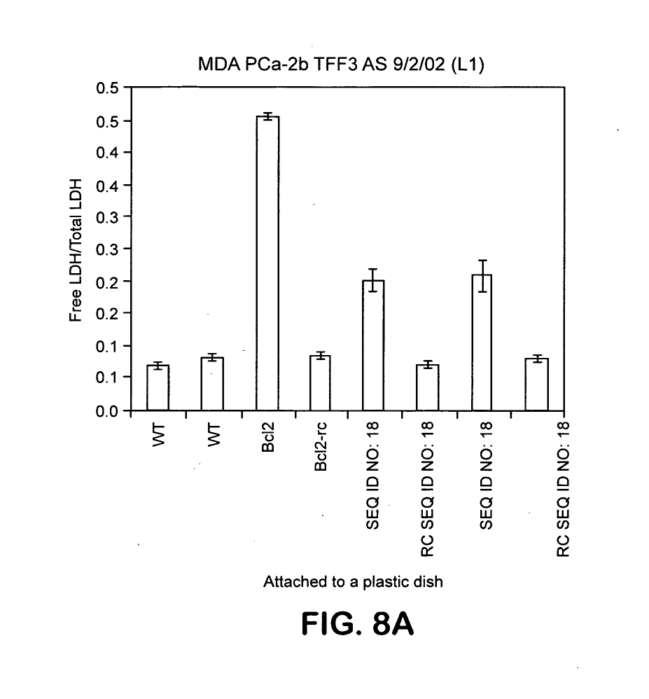

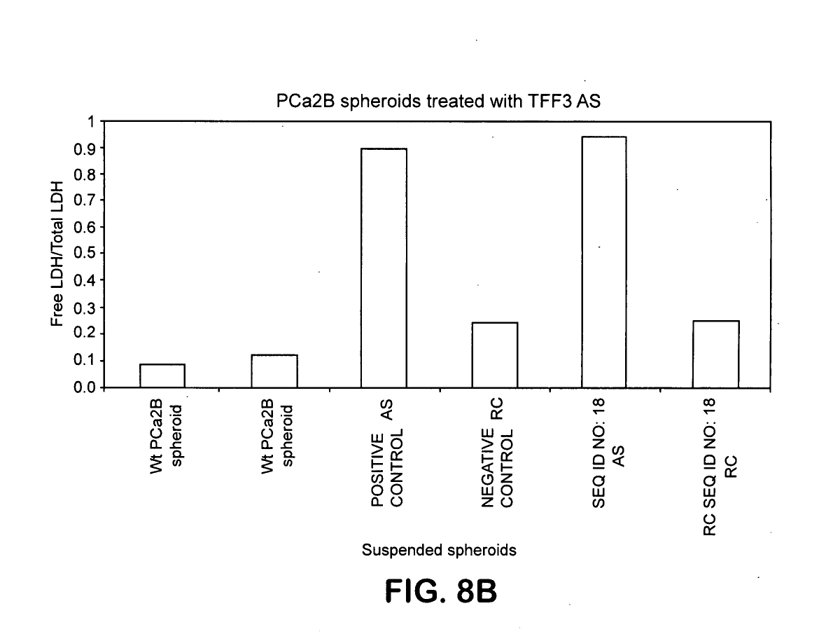

Cytotoxic effects were also observed in prostate cancer cells (Pca2B) in different culture conditions. The results are shown in FIG. 8. Similar results were obtained for prostate cancer cell lines CU145 and 22Rv1. A weaker effect was observed in PC3 and LNCaP cells. An antisense oligonucleotide known to inhibit expression of TFF3 and its reverse sequence serve as controls.

Normal Cells

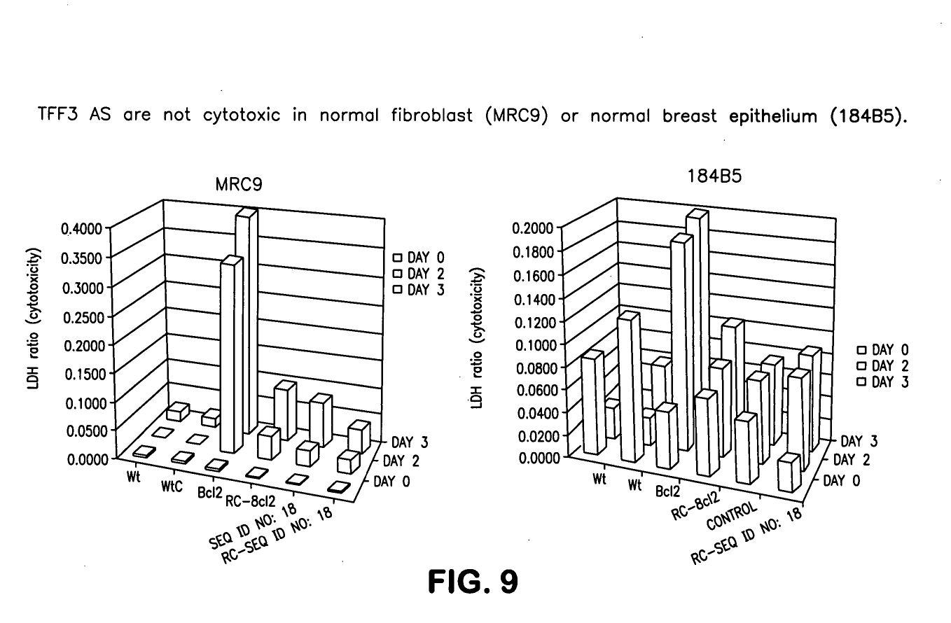

Normal fibroblast (MRC9) and normal breast epithelial cells (184B5) that do not express TFF3 were used as control cells to test for non-specific cytotoxic effects of the AS oligonucleotides. Results are depicted in FIG. 9. These results indicate that the effects of TFF3 AS are specific to cells expressing TFF3 and not due to general toxicity of the AS oligonucleotides.

### Example 9

### Effect of Gene Expression on Cell Migration

The effect of gene expression on the inhibition of cell migration can be assessed in LNCaP, PC3, 22Rv1, MDA-PCA-2b, or DU145 prostate cancer cells using static endothelial cell binding assays, non-static endothelial cell binding assays, and transmigration assays.

For the static endothelial cell binding assay, antisense oligonucleotides are prepared as described above (see Examples 4 and 5). Two days prior to use, prostate cancer cells (CaP) are plated and transfected with antisense oligonucleotide as described above (see Examples 4 and 5). On the day before use, the medium is replaced with fresh medium, and on the day of use, the medium is replaced with fresh medium containing 2 μM CellTracker green CMFDA (Molecular Probes, Inc.) and cells are incubated for 30 min. Following incubation, CaP medium is replaced with fresh medium (no CMFDA) and cells are incubated for an additional 30-60 min. CaP cells are detached using CMF PBS/2.5 mM EDTA or trypsin, spun and resuspended in DMEM/1% BSA/10 mM HEPES pH 7.0. Finally, CaP cells are counted and resuspended at a concentration of 1×10⁶ cells/ml.

Endothelial cells (EC) are plated onto 96-well plates at 40-50% confluence 3 days prior to use. On the day of use, EC are washed 1× with PBS and 50λ) DMDM/1% BSA/10 mM HEPES pH 7 is added to each well. To each well is then added 50 K (50λ) CaP cells in DMEM/1% BSA/10 mM HEPES pH 7. The plates are incubated for an additional 30 min and washed 5× with PBS containing Ca⁺⁺ and Mg⁺⁺. After the final wash, 100 μL PBS is added to each well and fluorescence is read on a fluorescent plate reader (Ab492/Em 516 nm).

For the non-static endothelial cell binding assay, CaP are prepared as described above. EC are plated onto 24-well plates at 30-40% confluence 3 days prior to use. On the day of use, a subset of EC are treated with cytokine for 6 hours then washed 2× with PBS. To each well is then added 150-200K CaP cells in DMEM/1% BSA/10 mM HEPES pH 7. Plates are placed on a rotating shaker (70 RPM) for 30 min and then washed 3× with PBS containing Ca⁺⁺ and Mg⁺⁺. After the final wash, 500 μL PBS is added to each well and fluorescence is read on a fluorescent plate reader (Ab492/Em 516 nm).

For the transmigration assay, CaP are prepared as described above with the following changes. On the day of use, CaP medium is replaced with fresh medium containing 5 μM CellTracker green CMFDA (Molecular Probes, Inc.) and cells are incubated for 30 min. Following incubation, CaP medium is replaced with fresh medium (no CMFDA) and cells are incubated for an additional 30-60 min. CaP cells are detached using CMF PBS/2.5 mM EDTA or trypsin, spun and resuspended in EGM-2-MV medium. Finally, CaP cells are counted and resuspended at a concentration of 1×10⁶ cells/ml.

EC are plated onto FluorBlok transwells (BD Biosciences) at 30-40% confluence 5-7 days before use. Medium is replaced with fresh medium 3 days before use and on the day of use. To each transwell is then added 50K labeled CaP. 30 min prior to the first fluorescence reading, 10 μg of FITC-dextran (10 K MW) is added to the EC plated filter. Fluorescence is then read at multiple time points on a fluorescent plate reader (Ab492/Em 516 nm).

Those antisense oligonucleotides that result in inhibition of binding of LNCaP, PC3, 22Rv1, MDA-PCA-2b, or DU145 prostate cancer cells to endothelial cells indicate that the corresponding gene likely plays a role in the production or maintenance of the cancerous phenotype in cancerous prostate cells. Those antisense oligonucleotides that result in inhibition of endothelial cell transmigration by LNCaP, PC3, 22Rv1, MDA-PCA-2b, or DU145 prostate cancer cells indicate that the corresponding gene likely plays a role in the production or maintenance of the cancerous phenotype in cancerous prostate cells.

### Example 10

### Effect of Gene Expression on Colony Formation

The effect of gene expression upon colony formation of SW620 cells, SKOV3 cells, MD-MBA-231 cells, LNCaP cells, PC3 cells, 22Rv1 cells, MDA-PCA-2b cells, and DU145 cells can be tested in a soft agar assay. Soft agar assays are conducted by first establishing a bottom layer of 2 ml of 0.6% agar in media plated fresh within a few hours of layering on the cells. The cell layer is formed on the bottom layer by removing cells transfected as described above from plates using 0.05% trypsin and washing twice in media. The cells are counted in a Coulter counter, and resuspended to 10⁶ per ml in media. 10 μl aliquots are placed with media in 96-well plates (to check counting with WST1), or diluted further for the soft agar assay. 2000 cells are plated in 800 μl 0.4% agar in duplicate wells above 0.6% agar bottom layer. After the cell layer agar solidifies, 2 ml of media is dribbled on top and antisense or reverse control oligo (produced as described in Example 3) is added without delivery vehicles. Fresh media and oligos are added every 3-4 days. Colonies form in 10 days to 3 weeks. Fields of colonies are counted by eye. Wst-1 metabolism values can be used to compensate for small differences in starting cell number. Larger fields can be scanned for visual record of differences.

Those antisense oligonucleotides that result in inhibition of colony formation of SW620 cells indicate that the corresponding gene plays a role in production or maintenance of the cancerous phenotype in cancerous colon cells. Those antisense oligonucleotides that inhibit colony formation in SKOV3 cells represent genes that play a role in production or maintenance of the cancerous phenotype in cancerous breast cells. Those antisense oligonucleotides that result in inhibition of colony formation of MDA-MB-231 cells indicate that the corresponding gene plays a role in production or maintenance of the cancerous phenotype in cancerous ovarian cells. Those antisense oligonucleotides that inhibit colony formation in LNCaP, PC3, 22Rv1, MDA-PCA-2b, or DU145 cells represent genes that play a role in production or maintenance of the cancerous phenotype in cancerous prostate cells.

### Example 11

### Antisense Inhibition of Anchorage-Independent Growth of Cancer Cells

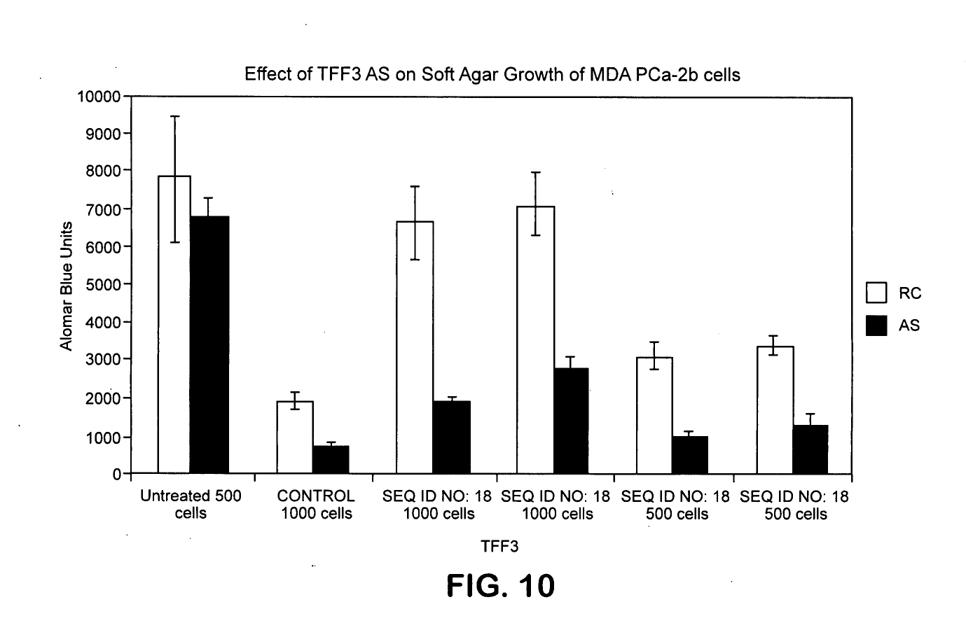

The prostate cancer cell line MDA Pca-2b was transfected with a TFF3 antisense oligonucleotide (as indicated in FIG. 10; see Examples 4 and 5 for description of antisense preparation). One thousand or 500 cells per well (as indicated in FIG. 10) were cultured in a 96-well plate and grown in suspension for 7 days in soft agar containing media. The growth of soft-agar cell colonies was measured by the incorporation of Alamar blue by the cells. The results indicate that both control AS (C79-7) and TFF3 AS oligonucleotides (but not the respective RC oligonucleotides) significantly inhibited the anchorage independent growth of this prostate cancer cell line. Similar results were obtained for the prostated cancer cell line PC3.

### Example 12

### Functional Analysis of Gene Products Differentially Expressed in Prostate Cancer in Patients

The gene products (such as TFF3) of sequences of a gene differentially expressed in cancerous cells can be further analyzed to confirm the role and function of the gene product in tumorigenesis, e.g., in promoting or inhibiting development of a metastatic phenotype. For example, the function of gene products corresponding to genes identified herein can be assessed by blocking function of the gene products in the cell. For example, where the gene product is secreted or associated with a cell surface membrane, blocking antibodies can be generated and added to cells to examine the effect upon the cell phenotype in the context of, for example, the transformation of the cell to a cancerous, particularly a metastatic, phenotype. In order to generate antibodies, a clone corresponding to a selected gene product is selected, and a sequence that represents a partial or complete coding sequence is obtained. The resulting clone is expressed, the polypeptide produced isolated, and antibodies generated. The antibodies are then combined with cells and the effect upon tumorigenesis assessed.

Where the gene product of the differentially expressed genes identified herein exhibits sequence homology to a protein of known function (e.g., to a specific kinase or protease) and/or to a protein family of known function (e.g., contains a domain or other consensus sequence present in a protease family or in a kinase family), then the role of the gene product in tumorigenesis, as well as the activity of the gene product, can be examined using small molecules that inhibit or enhance function of the corresponding protein or protein family.

Additional functional assays include, but are not necessarily limited to, those that analyze the effect of expression of the corresponding gene upon cell cycle and cell migration. Methods for performing such assays are well known in the art.

### Example 13

### Contig Assembly and Additional Gene Characterization

The sequences of the polynucleotides provided in the present invention (e.g., TFF3) can be used to extend the sequence information of the gene to which the polynucleotides correspond (e.g., a gene, or mRNA encoded by the gene, having a sequence of the polynucleotide described herein). This expanded sequence information can in turn be used to further characterize the corresponding gene, which in turn provides additional information about the nature of the gene product (e.g., the normal function of the gene product). The additional information can serve to provide additional evidence of the gene product's use as a therapeutic target, and provide further guidance as to the types of agents that can modulate its activity.

In one example, a contig is assembled using a sequence of a polynucleotide of the present invention, which is present in a clone. A “contig” is a contiguous sequence of nucleotides that is assembled from nucleic acid sequences having overlapping (e.g., shared or substantially similar) sequence information. The sequences of publicly-available ESTs (Expressed Sequence Tags) and the sequences of various clones from several cDNA libraries synthesized at Chiron can be used in the contig assembly.

The contig is assembled using the software program Sequencher, version 4.05, according to the manufacturer's instructions and an overview alignment of the contiged sequences is produced. The sequence information obtained in the contig assembly can then be used to obtain a consensus sequence derived from the contig using the Sequencher program. The consensus sequence is used as a query sequence in a TeraBLASTN search of the DGTI DoubleTwist Gene Index (DoubleTwist, Inc., Oakland, Calif.), which contains all the EST and non-redundant sequence in public databases.

Through contig assembly and the use of homology searching software programs, the sequence information provided herein can be readily extended to confirm, or confirm a predicted, gene having the sequence of the polynucleotides described in the present invention. Further the information obtained can be used to identify the function of the gene product of the gene corresponding to the polynucleotides described herein. While not necessary to the practice of the invention, identification of the function of the corresponding gene, can provide guidance in the design of therapeutics that target the gene to modulate its activity and modulate the cancerous phenotype (e.g., inhibit metastasis, proliferation, and the like).

### Example 14

### In Vivo Testing of TFF3 Neutralizing Agents

TFF3 neutralizing agents can be tested for anti-tumor activity in animal models according to procedures known in the art for pre-clinical assessment of drug candidates. Suitable animal models include TRAMP mice (available from NCI-Frederick Mouse Models of Human Cancer Consortium Repository or The Jackson Laboratory) for prostate cancer and Min mice (available from The Jackson Laboratory) for colon cancer. Animal models for other cancers are well known in the art.

### Example 15

### TFF3 Epitopes

Linear epitopes of TFF3 for antibody recognition and preparation can be identified by any of numerous methods known in the art. Some example methods include probing antibody-binding ability of peptides derived from the amino acid sequence of the antigen. Binding can be assessed by using BIACORE or ELISA methods. Other techniques include exposing peptide libraries on planar solid support (“chip”) to antibodies and detecting binding through any of multiple methods used in solid-phase screening. Additionally, phage display can be used to screen a library of peptides with selection of epitopes after several rounds of biopanning. Suitable antibody neutralizing agents according to the present invention can recognize linear or conformational epitopes, or combinations thereof.

Table 5 below provides regions of TFF3 that have been identified as linear epitopes suitable for recognition by anti-TFF3 antibodies.

TABLE 5

| | Mapped | | | | |
|---|---|---|---|---|---|
| | amino acid | Mapped | | | |
| | sequence | epitope | | SEQ ID | |
| ECD name | location | location | Length | NO: | Sequence |
| TFF3#1 | 27-34 | 27-33 | 8-mer | 20 | AVPAKDRV |
| TFF3#1 | 27-34 | 28-34 | 8-mer | 21 | VPAKDRVD |
| TFF3#1 | 27-34 | 27-34 | 9-mer | 22 | AVPAKDRVD |
| TFF3#2 | 36-44 | 36-43 | 8-mer | 23 | GYPHVTPK |
| TFF3#2 | 36-44 | 37-44 | 8-mer | 24 | YPHVTPKE |
| TFF3#2 | 36-44 | 36-44 | 9-mer | 25 | GYPHVTPKE |
| TFF3#3 | 63-71 | 63-70 | 8-mer | 26 | FKPLQEAE |
| TFF3#3 | 63-71 | 64-71 | 8-mer | 27 | KPLQEAEC |
| TFF3#3 | 63-71 | 63-71 | 9-mer | 28 | FKPLQEAEC |

### Example 16

### Polyclonal Antibodies Against TFF3 Inhibit Tumor Cell Growth

A. Generation of Polyclonal Antibodies

New Zealand albino rabbits were anesthetized and immunized on Day 0 and Day 28 using a DNA expression vector encoding human TFF3. Specifically, for each immunization, 0.6 mg of the DNA expression vector was injected intramuscularly per rabbit, and a mild electric current was briefly applied to the injection site to stimulate uptake of the DNA by muscle cells in the area. The rabbits were then boosted monthly by intramuscular injection of recombinant human TFF3 protein emulsified in either MF-59 adjuvant or Incomplete Freund's Adjuvant. Blood samples were taken 14 days after each immunization, and serum generated from the blood samples was tested in an ELISA assay to determine the titer of the antibody response against the recombinant human TFF3 protein. The sera were then fractionated by chromatography over a Protein A column in order to purify the IgG antibody component in each sample.

B. SW620 Proliferation Assay

In order to test whether the rabbit polyclonal antibodies could affect the survival of cancer cells, serial dilutions of the purified IgGs obtained from the immunized rabbits were added to a cancer cell line (SW620) that had been shown by Western blotting to secrete TFF3 protein. For this test, the SW620 cells were first seeded into 96-well plates at a density of 600 cells/well in growth medium containing 10% fetal bovine serum (FBS). Twenty-four hours later (Day 0), the medium was removed, and fresh growth medium was added that contained 1% FBS and various concentrations of the IgGs from immunized or preimmune rabbits. Each IgG concentration was tested in quadruplicate wells. On Day 4, the medium was again removed from the plates, and fresh medium containing 1% FBS and aliquots of the respective IgG fraction was re-added to the wells. The relative number of cells in each well was measured on Days 0,1,4,5,6 and 7 using the “Cell Titer One Solution Cell Proliferation Assay” (Promega).

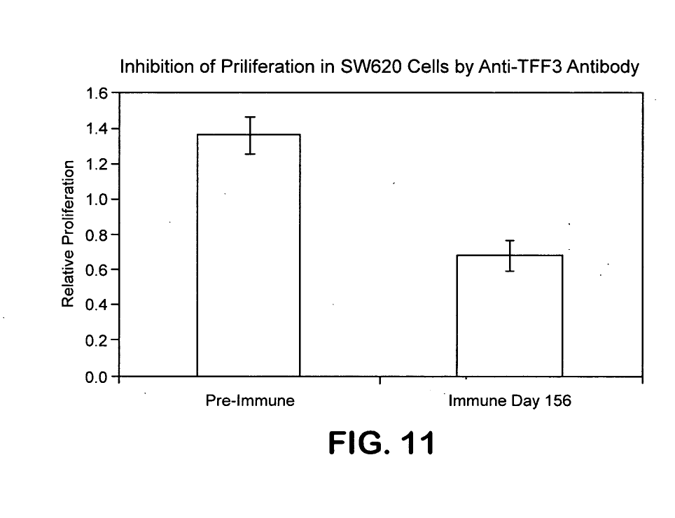

The results of one such test are shown in FIG. 11 for IgG antibodies obtained from one of the immunized rabbits (Rabbit 707). The graph compares the amount of proliferation detected at Day 7 in wells containing IgG taken from the rabbit either prior to the first immunization (“Pre-Immune”), or after several rounds of immunization (“Immune Day 156”). The wells containing the “Immune Day 156” IgG showed significantly less proliferation than those containing the “Pre-Immune” IgG.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating or preventing cancer comprising administering to a mammal afflicted with said cancer or predisposed to said cancer a therapeutically effective amount of a TFF3 neutralizing agent.

2. The method of claim 1 wherein said TFF3 neutralizing agent comprises a nucleic acid.

3. The method of claim 1 wherein said cancer is breast, colon, prostate, ovarian, or gastric cancer.

4. The method of claim 1 wherein TFF3 is differentially expressed in cells of said cancer.

5. The method of claim 1 wherein said neutralizing agent comprises an antisense molecule.

6. The method of claim 5 wherein said antisense molecule comprises or overlaps a sequence of any of SEQ ID NOs: 5-19.

7. The method of claim 1 wherein said neutralizing agent comprises an RNAi molecule.

8. The method of claim 7 wherein said RNAi molecule comprises or overlaps with a sequence of any of SEQ ID NOS: 5-19.

9. The method of claim 1 wherein said TFF3 neutralizing agent comprises an antibody which specifically binds to TFF3.

10. The method of claim 1 wherein said cancer is not colon or prostate cancer

11. A method of treating cancer comprising administering to a mammal afflicted with said cancer or predisposed to said cancer a therapeutically effective amount of a TFF3 neutralizing agent and providing said mammal with a traditional cancer treatment.

12. The method of claim 11 wherein said traditional cancer treatment is chemotherapy.

13. The method of claim 11 wherein said traditional cancer treatment is hormone ablation therapy.

14. The method of claim 13 wherein said hormone is an androgen.

15. The method of claim 11 wherein said TFF3 neutralizing agent comprises an antibody, nucleic acid, antisense molecule, or RNAi molecule.

16. A method of inducing apoptosis in a cell comprising contacting said cell with a TFF3 neutralizing agent.

17. The method of claim 16 wherein said cell is mammalian.

18. The method of claim 16 wherein said cell is cancerous.

19. The method of claim 16 wherein said cell is a breast, prostate, colon, ovarian, or gastric cell.

20. The method of claim 16 wherein said TFF3 neutralizing agent comprises an antibody, nucleic acid, antisense molecule, or RNAi molecule.

21. A method of reducing tumor volume, slowing tumor growth, or preventing tumor growth comprising contacting said tumor with a TFF3 neutralizing agent.

22. The method of claim 21 wherein said TFF3 neutralizing agent comprises an antibody, nucleic acid, antisense molecule, or RNAi molecule.

23. The method of claim 21 wherein said tumor comprises cells in which TFF3 is differentially expressed.

24. A method of modulating at least one physiological effect associated with expression of TFF3 in a cell comprising contacting said cell with a TFF3 neutralizing agent.

25. The method of claim 24 wherein said physiological effect is increased cell motility or resistance to apoptosis.

26. A method of inhibiting the migration, adhesion, or proliferation of a cell comprising contacting said cell with a TFF3 neutralizing agent.

27. A method of reducing invasiveness of a cancer cell comprising contacting said cancer cell with a TFF3 neutralizing agent.

28. A method of modulating TFF3 expression in a cell comprising contacting said cell with a TFF3 neutralizing agent.

29. A method of detecting TFF3 in a biological sample comprising contacting said sample with a TFF3 neutralizing agent and detecting binding between said neutralizing agent and TFF3 in said sample.

30. The method of claim 29 wherein said TFF3 neutralizing agent comprises a detectable label.

31. A method for detecting the presence of cancer in a biological sample comprising:
contacting said biological sample with a TFF3 neutralizing agent and detecting evidence of differential expression of TFF3 in said biological sample, wherein evidence of differential expression of TFF3 is indicative of the presence of cancer.

32. The method of claim 31 wherein said detecting comprises comparing the results of said contacting with a control.

33. The method of claim 31 wherein said TFF3 neutralizing agent comprises a detectable label.

34. A method for determining the susceptibility of a patient to a TFF3 neutralizing agent comprising detecting evidence of differential expression of TFF3 in said patient's cancer sample, wherein evidence of differential expression of TFF3 is indicative of the patient's susceptibility to said TFF3 neutralizing agent.

35. The method of claim 34 wherein said evidence of differential expression of TFF3 is upregulation of TFF3 in said patient's cancer sample.

36. The method of claim 34 wherein said patient's cancer sample is from breast, prostate, colon, ovarian, or gastric tissue.

37. A method for assessing the progression of cancer in a patient comprising comparing the expression of TFF3 in the patient at a first time point to the expression of TFF3 at a second time point, wherein increased expression of TFF3 at the second time point relative to the first time point is indicative of progression of said cancer.

38. The method of claim 37 wherein said increased expression of TFF3 is increased expression of at least about 25%.

39. A method for detecting an increased risk of metastasis of a cancer in a patient comprising comparing the expression of TFF3 in the patient at a first time point to the expression of TFF3 at a second time point, wherein increased expression of TFF3 at the second time point relative to the first time point is indicative of said increased risk of metastasis.

40. An antisense molecule that modulates expression of TFF3.

41. The antisense molecule of claim 40 wherein said antisense molecule comprises or overlaps a sequence of any of SEQ ID NOS: 5-19.

42. The antisense molecule of claim 40 comprising any of SEQ ID NOS: 5-19.

43. An RNAi molecule that modulates expression of TFF3.

44. The RNAi molecule of claim 43 wherein said RNAi molecule comprises or overlaps a sequence of any of SEQ ID NOS: 5-19.

45. A composition comprising an antisense molecule of claim 40 and a pharmaceutically acceptable carrier.

46. A composition comprising an RNAi molecule of claim 43 and a pharmaceutically acceptable carrier.

47. An isolated anti-TFF3 antibody, wherein said antibody recognizes at least one region of TFF3 sequence corresponding to SEQ ID NO: 20, 21, 22, 23, 24, 25, 26, 27 or 28.

48. The antibody of claim 47 wherein said antibody is produced by a process comprising:
a) synthesizing a library of antibodies on phage;
b) panning the library against a sample by bringing the phage into contact with a composition comprising at least one region of TFF3 sequence corresponding to SEQ ID NO: 20, 21, 22, 23, 24, 25, 26, 27 or 28;
c) isolating phage which bind said composition, wherein the antibody is characterized by its ability to bind to at least one region of TFF3 sequence corresponding to SEQ ID NO: 20, 21, 22, 23, 24, 25, 26, 27 or 28 with a binding affinity of at least 10⁸ l/; and
d) analyzing the isolated phage to determine a sequence encoding an amino acid sequence to which the at least one region of TFF3 sequence corresponding to SEQ ID NO: 20, 21, 22, 23, 24, 25, 26, 27 or 28 binds.

49. The antibody of claim 47 wherein said antibody is a monoclonal antibody.

50. The antibody of claim 47 wherein said antibody is a polyclonal antibody.

51. The antibody of claim 47 wherein said antibody is a chimeric antibody.

52. The antibody of claim 47 wherein said antibody is a humanized antibody.

53. The antibody of claim 47 wherein said antibody is a single-chain antibody.

54. The antibody of claim 47 wherein said antibody is a Fab fragment.

55. The antibody of claim 47 wherein said antibody is labeled.

56. The antibody of claim 55 wherein said label is an enzyme, radioisotope, or fluorophore.

57. The antibody of claim 47 wherein the binding affinity of said antibody is less than about 1×10⁵ Kₐ for a polypeptide other than TFF3.

58. An isolated cell that produces the antibody of claim 47.

59. A hybridoma that produces the antibody of claim 47.

60. A composition comprising the anti-TFF3 antibody of claim 47 and a pharmaceutically acceptable carrier.

61. A method of treating or preventing cancer comprising administering to a mammal afflicted with said cancer or predisposed to said cancer a therapeutically effective amount of the antibody of claim 47.

62. A method of treating cancer comprising administering to a mammal afflicted with said cancer or predisposed to said cancer a therapeutically effective amount of the antibody of claim 47 and providing said mammal with a traditional cancer treatment.

63. The method of claim 62 wherein said traditional cancer treatment is chemotherapy.

64. The method of claim 62 wherein said traditional cancer treatment is hormone ablation therapy.

65. The method of claim 64 wherein said hormone is an androgen.

66. A method of inducing apoptosis in a cell comprising contacting said cell with the antibody of claim 47.

67. The method of claim 66 wherein said cell is mammalian.

68. The method of claim 66 wherein said cell is cancerous.

69. A method of reducing tumor volume, slowing tumor growth, or preventing tumor growth comprising contacting said tumor with the antibody of claim 47.

70. The method of claim 69 wherein said tumor comprises cells in which TFF3 is differentially expressed.

71. A method of modulating at least one physiological effect associated with expression of TFF3 in a cell comprising contacting said cell with the antibody of claim 47.

72. The method of claim 71 wherein said physiological effect is increased cell motility or resistance to apoptosis.

73. A method of inhibiting the migration, adhesion, or proliferation of a cell comprising contacting said cell with the antibody of claim 47.

74. A method of reducing invasiveness of a cancer cell comprising contacting said cancer cell with the antibody of claim 47.

75. A method of detecting TFF3 in a biological sample comprising contacting said sample with the antibody of claim 47 and detecting binding between said antibody and TFF3 in said sample.

76. The method of claim 75 wherein said antibody comprises a detectable label.

77. A method for detecting the presence of cancer in a biological sample comprising: contacting said biological sample with the antibody of claim 47 and detecting evidence of differential expression of TFF3 in said biological sample, wherein evidence of differential expression of TFF3 is indicative of the presence of cancer.

78. The method of claim 77 wherein said detecting comprises comparing the results of said contacting with a control.

79. The method of claim 77 wherein said antibody comprises a detectable label.

80. An isolated polypeptide comprising three or fewer amino acid sequences selected from SEQ ID NOS: 20, 21, 22, 23, 24, 25, 26, 27 or 28.

81. The polypeptide of claim 80 wherein said polypeptide is from about 8 to about 80 amino acids in length.

82. The peptide of claim 80 wherein said polypeptide binds specifically to an anti-TFF3 antibody.

83. A method of using an antibody to detect differential expression of TFF3 in a sample comprising:
a) combining the antibody of claim 47 with said sample under conditions which allow the formation of antibody:TFF3 complexes;
b) measuring the amount of said complexes; and
c) comparing the amount of said complexes to a control, wherein elevated levels of complex in said sample indicates differential expression of TFF3.

84. An isolated epitope-bearing fragment of the polypeptide of SEQ ID NO: 1-4.

85. The epitope-bearing fragment of claim 84, which consists of between about 6 and about 20 contiguous amino acids of SEQ ID NO: 1-4.

86. The epitope-bearing fragment of claim 85, which consists of about 10 contiguous amino acids of SEQ ID NO: 1-4.

87. The epitope-bearing fragment of claim 84, which consists of between about 6 and about 20 contiguous amino acids of SEQ ID NO:2.

88. The epitope-bearing fragment of claim 85, which consists of about 10 contiguous amino acids of SEQ ID NO:2.

89. The epitope-bearing fragment of claim 85 which comprises SEQ ID NO: 20, 21, 22, 23, 24, 25, 26, 27 or 28.

90. An isolated anti-TFF3 antibody which is obtained by immunization of a subject with the epitope-bearing fragment of claim 85.

91. A pharmaceutical composition comprising the antibody of claim 47 and a pharmaceutically acceptable carrier.

92. The pharmaceutical composition of claim 91 wherein the antibody neutralizes TFF3.

93. A method for generating an antibody for the treatment of cancer comprising identifying an antibody that binds to and neutralizes TFF3, and expressing the antibody in a recombinant expression host cell.

94. A pharmaceutical composition comprising an antibody that binds to and neutralizes TFF3, wherein the antibody was generated using a recombinant host cell, and a pharmaceutically acceptable carrier.

95. The pharmaceutical composition of claim 94 wherein the recombinant host cell is selected from the group consisting of Chinese Hamster Ovary cell, myeloma cell and bacterial host cell.
